(19) United States
(12) Patent Application Publication
(10) Pub. No.: US 2010/0151468 A1
(43) Pub. Date: Jun. 17, 2010

(54) EPIGENETIC BIOMARKERS FOR EARLY DETECTION, THERAPEUTIC EFFECTIVENESS, AND RELAPSE MONITORING OF CANCER

(76) Inventors: Manel Esteller, Madrid (ES); Fraga Mario, Madrid (ES); Ballestar Esteban, Madrid (ES); Guerrero-Preston Rafael, Baltimore, MD (US)

Correspondence Address: Pearl Cohen Zedek Latzer, LLP, 1500 Broadway, 12th Floor, New York, NY, 10036, US

(21) Appl. No.: 12/595,166

(22) PCT Filed: Apr. 11, 2008
(86) PCT No.: PCT/US08/04736

§ 371 Date: Feb. 25, 2010

Related U.S. Application Data
(60) Provisional application No. 60/911,269, filed on Apr. 11, 2007.

(51) Int. Cl. C12Q 1/68; G01N 33/574; G01N 33/48

(52) U.S. Cl. 435/6; 436/64; 436/94

(57) ABSTRACT

The present invention provides methods of detection, including early detection, for cancer or other diseases and normal physiologic processes mediated by global epigenetic changes, by using one or more of the following biomarkers: a global DNA methylation index, a global histone H4 acetylation index, and a global histone H4 trimethylation index. These methods are useful for, among other things, assessing the effectiveness of treatment, monitoring relapse, and clinical staging of cancer and other chronic as well as acute diseases. These methods are also useful for among other things monitoring the effectiveness of strategies and therapies used to modify lifestyle and contextual effects to prevent disease, foster wellness and enable health promotion.

### STATEMENT REGARDING FEDERALLY SPONSORED RESEARCH

This work was supported in part by funds from the federal government (NCI grant number 5T32CA009529-20, NIA grant number 2P30AG-15294 and NCMHD grant number 5S21MD008130-02). Therefore, the federal government has certain rights in this invention.

### FIELD OF THE INVENTION

This invention relates to diagnostic, screening, and early detection methods for cancer, which can also be used to monitor therapeutic effectiveness and relapse monitoring in cancer and other pathological and physiological processes.

### BACKGROUND OF THE INVENTION

The inheritance of information based on gene expression levels is known as epigenetics, as opposed to genetics, which refers to information transmitted on the basis of gene sequence. Cancer, which includes any malignant neoplastic disease, including but not limited to solid tumors and hematologic malignancies, as well as premalignant conditions, are epigenetic diseases characterized by the generation of aberrant patterns of DNA methylation and histone modifications with dramatic consequences in gene expression and architectural organization of genomic information (Esteller, 2008, Ballestar, 2008). Epigenetic events represent important mechanisms by which gene expression is selectively activated or inactivated leading to functional and biological alterations, which accumulate during aging and are important in tumorigenesis. (Fraga, 2007a) In utero exposures can lead to life-course imprinting in the offspring and potentially modify disease susceptibility and risk (Sinclair, 2007). The epigenome reproduced during mitosis and can be inherited across generations. The innate plasticity of the epigenome also enables it to be reprogrammed by social, chemical, biological and physical factors (Dolinoy, 2008).

Emerging evidence indicates that various epigenetic alterations common to most types of cancer, such as global histone modifications and DNA hypomethylation, are also observed in other chronic diseases (Wilson, 2007). In many cases, epigenetic modifications are reversible, thus providing an opportunity to reverse the chronic disease process and understand the impact of lifestyle choices on chronic disease susceptibility and risk (Herranz, 2007).

The stability of our genome and correct gene expression is maintained to a great extent by a perfectly preestablished pattern of DNA methylation and histone modifications. In cancer and other chronic diseases this scenario breaks down due a sudden loss of global methylation associated with histone modifications which lead to genomic instability, chromosomal rearrangements, activation of transposable elements and retroviruses, microsatellite instability and aberrant gene expression (Guerrero-Preston, 2007, Esteller, 2006a). In cancer an interesting gene-specific phenomenon following global DNA hypomethylation has been widely studied whereby the regulatory regions (CpG islands) of certain tumor suppressor genes (such as BRCA1, hMLH1, p16^{INK4a}, and VHL) become hypermethylated, inactivating the gene as a consequence, whilst the regulatory regions of proto-oncogenes become hypomethylated thus leading to transcriptional activation of the oncogene (Esteller, 2007a, Esteller, 2006b). Thus global DNA hypomethylation is usually seen together with gene-specific hyper and hypomethylation in cancer and other chronic diseases (Ehlrich, 2006). The global methylcytosine content of a large collection of normal tissues and tumors has been studied to begin to understand this mechanism in cancer and other diseases (Hoffmann, 2005).

The human epigenome is dynamic, not only throughout the cell cycle and during mitotic divisions, but also in its response to environmental factors, which can be critical in development and during aging (Fraga, 2007b). Transient and fixed epigenetic modifications continually modulate the normal human epigenome throughout the life course in response to endogenous and exogenous stimuli. The epigenome serves as an interface between the dynamic environment and the inherited static genome, configured during development to shape the diversity of gene expression programs in the different cell types of the organism by a highly organized process. It is has been shown that exposure to physical, biological and chemical factors, as well as exposure to social behavior, such as maternal care, modifies the epigenome (Szyf, 2008). Therefore exposures to different environmental agents throughout the life course may lead to interindividual phenotypic diversity, as well as differential susceptibility to disease and behavioral pathologies.

The responses of the epigenome to environmental exposures throughout the life-course are not just aberrations leading to pathology but a biological mechanism that serves as a medium for the adaptability of the genome to altered environments during life. External exposures, physical, chemical, biological and physical exposures received at different levels of social organization lead to changes in the extracellular environment of developing or mature somatic cells, activating signaling pathways, which link extracellular environmental exposures and epigenetic machineries (Szyf, 2007).

The epigenomic machineries are the biological substrate that serves as a mediator between endogenous and exogenous stimuli at different levels of biological organization and the resultant gene expression, which leads to adaptive or reactive responses to said stimuli. The interaction between the internal or external environment and the epigenome is exposure, tissue and cell specific. Therefore environmental stimuli lead to changes in gene expression levels by interacting with epigenetic machineries without altering the sequence of DNA bases (Dolinoy, 2006). This interaction leads to a modulation in biological and/or psychological processes (Szyf et al, 2007; Weaver et al, 2006) that adjust gene expression, in transient and permanent fashion through-out the life-course: from womb to grave. The interaction between non-genotoxic environmental stressors and environmental health promoters and the epigenome occurs at different pathways and intersections of cellular, organ, systemic and bodily functions; from memory formation and synaptic plasticity (Miller et al, 2007; Miller and Sweat, 2007) to adaptation to changing environments (Weaver, 2007).

Non-genotoxic exposures to, for example, DNA and RNA viruses, alcohol, cigarette smoke, obesity, diabetes, poor diet and sedentary life-styles, all risk factors that have been associated to cancer, lead to extracellular changes, which activate signaling pathways associated to histones modifications and cause changes in global DNA methylation in the background of normal and pathogenic cellular activity. The global DNA methylation changes lead to structural chromosomal instability at different repetitive sequences, aberrant gene expression, and loss of imprinting (Guerrero-Preston, 2007).

In the earlier days of cancer research, stepwise and orderly progression of genetic alterations causing activation of oncogenes and inactivation of tumor suppressor genes was considered to be the molecular framework responsible for multistage carcinogenesis in humans. However, genetic events alone may not explain the entire process of carcinogenesis: only a few genetic alterations are known to be responsible, especially in the earlier, precancerous stages. Cancer is now understood as an epigenetic disease characterized by the breakdown of DNA methylation and histone modification patterns, which lead to the genetic alterations observed in sporadic cancers. Emerging evidence indicates that various epigenomic alterations, such as global histone modifications and global DNA hypomethylation, are marks common to most types of cancer. These global marks represent a non-specific continuous surrogate measurement of genome-wide and gene-specific changes that arise as adaptive, tissue and cell specific, responses to environmental insult throughout out human developmental stages, from fertilization until death. The origin of cancer lies then in the epigenome and epigenetic biomarkers can be used to detect, diagnose and manage solid and hematological tumors (Lujambio, 2007a Meaney, 2005).

The epigenetic theory of oncogenesis does not contradict the monoclonal origin of tumors, proposed by Fialkow in 1979 and still considered the canonical theory of oncogenesis, in as much as it provides an explanation for the molecular modifications that need to happen before the known mutations that lead to clonal selection in cancer can occur. The consensus is that the vast majority of human tumors are monoclonal growths descended from single progenitor cells, which overcome the constraints imposed by multi-cellularity and development through several rounds of mutations and clonal selection. More recently it has been suggested that only a subset of solid and hematopoietic tumor cells have clonogenic capacity and are thus tumorigenic. These cells, referred to as cancer stem cells, are postulated to drive tumor growth, progression and metastasis in response to acute or chronic environmental insult, which in the case of sporadic cancers occurs during critical periods of development in utero and also later in life.

The number of accumulated mutations required to drive oncogenic processes argue against the monoclonal theory of oncogenesis, regardless whether the chain of gene-specific mutational events occurs in cancer stem cells or in cells modified by the tumor microenvironment. The epigenetic theory of oncogenesis provides the mechanistic explanation that links environmental exposures at the systemic and tumor micro-environments to the adaptive molecular changes that precede gene-specific mutations and clonal selection in cancer.

Successful strategies of early detection in cancer should be able to detect the difference between global and gene-specific epigenetic patterns in normal cells from those epigenetic patterns in cells with cancer or at risk of developing cancer. The global genome-wide and gene-specific changes to the epigenome in cancer, including DNA hypomethylation, hypermethylation, chromatin alterations and miRNA gene silencing, are made clear by a systematic examination of the cancer epigenome at the molecular level (Lujambio, 2007b, Esteller, 2007b).

DNA methylation, the most important epigenetic modification known, is a chemical modification of the DNA molecule itself, which is carried out by an enzyme called DNA methyltransferase. DNA methylation can directly switch off gene expression by preventing transcription factors binding to promoters. However, a more general effect is the attraction of methyl-binding domain (MBD) proteins. These are associated with further enzymes called histone deacetylases (HDACs), which function to chemically modify histones and change chromatin structure. Chromatin containing acetylated histones is open and accessible to transcription factors, and the genes are potentially active. Histone deacetylation causes the condensation of chromatin, making it inaccessible to transcription factors and the genes are therefore silenced (Eberharter, 2002), The link between histone deacetylation and DNA methylation was the finding that MeCP2 physically interacts with the transcriptional co-repressor protein Sin3A, and in so doing recruits a histone de-acetylase (HDAC) to chromatin that contains methylated DNA (Tycko, 2000, Studnicki, 2005).

Less attention has been focused on histones modifications in cancer cells. Post-translational modifications to histones H4 in a comprehensive panel of normal tissues, cancer cell lines and primary tumors were recently characterized. These changes appeared early and accumulated during the tumorigenic process, as shown in a mouse model of multistage skin carcinogenesis (Fraga, 2004). The losses occurred predominantly at the acetylated Lys16 and trimethylated Lys20 residues of histones H4 and were associated with the hypomethylation of DNA repetitive sequences. This data suggests that the global loss of monoacetylation and trimethylation of histones H4 is a common hallmark of human tumor cells (Fraga, 2005a). Therefore, loss of acetylated Lys16 and trimethylated Lys20 residues of histones H4 could also be used as early detection biomarkers of all human cancer.

Several mechanisms have been proposed to explain the alteration of global, genome-wide and gene-specific epigenetic patterns in cancer, which range from in-utero imprinting to a sequential accumulation of epigenetic changes associated with exposures to environmental stressors throughout the life-course (Fraga, 2005c). A recently published study by a co-inventor (Fraga, 2005a) examined the global and locus-specific differences in DNA methylation and histone acetylation of a large cohort of monozygotic twins. They found that, although twins are epigenetically indistinguishable during the early years of life, older monozygous twins exhibited remarkable differences in their overall content and genomic distribution of 5-methylcytosine DNA and histone acetylation, affecting their gene-expression portrait.

Experimental data suggest that genes involved in DNA methylation, histones modification and chromatin remodeling also become disrupted in cancer. Some of these will act as oncogenes, others as tumor-suppressor genes. Some will be altered by genetic lesions, others by epigenetic lesions (Esteller, 2006).

### SUMMARY OF THE INVENTION

The present invention relates to three epigenetic modifications that occur very early in the oncogenic process and have been identified as a hallmark of all human cancers. In certain embodiments, the epigenetic modifications are global DNA hypomethylation, global decrease in histone H4 methylation, and global decrease in histone H4 acetylation. When measured in a global assay, each of these modifications by themselves can be an informative biomarker for early detection of cancer, and together can improve specificity and sensitivity as an early detection tool according to different cancer sites/types characteristics. These global biomarkers can also be combined with genome-wide (Mund et al, 2006, Yang et al, 2004) and gene-specific biomarkers (Shen et al, 2007) to detect molecular changes associated to pathogenesis and disease. These biomarkers have been tested in DNA and histones extracted from tissue and bodily fluids (Wong et al, 2001; Lecomte et al, 2002) obtained from cells, clinical samples and case-control cohorts. (Zhang, et al, 2007; Seligson et al, 2005); These biomarkers can be further validated in studies with larger populations. In addition, these epigenetic biomarkers can be incorporated into early detection, clinical management and disease recurrence monitoring kits designed for effective measurement in but not limited to exfoliated cells obtained from blood, saliva, tears, urine, cervical smear, ductal lavage fluid, cerebrospinal fluid, lymph fluid, serosal fluid, bile and stool. These multiplexed kits can be used as part of an integrative trans-omics approach that combines additional global, genome-wide and gene-specific molecular markers to identify the initial epigenetic modifications observed in the transition from the normal to a diseased cell, and conversely the reversible epigenetic modifications observed in the transition from diseased or chronically challenged cells to healthier cells.

DNA methylation, the most important epigenetic modification known, plays a dual role in human cancer. Global hypomethylation, together with both hypomethylation (Kaneda et al, 2004) and hypermethylation of promoter regions, are fundamental aspects of human neoplasia (Baylin et al, 1998; Feinberg and Vogelstein 1983). Region-specific hypermethylation of CpG islands leads to the suppression of housekeeping and cell cycle control genes, as well as tumor suppressor and DNA repair genes, resulting in tumor growth and progression (Jones and Baylin, 2002). Global hypomethylation plays a causal role in tumor formation by promoting chromosomal instability, activation of proto-oncogenes, and loss of heterozygosity, all of which are highly correlated with tumorigenesis (Eden et al, 2003; Gaudet et al, 2003; Matsuzaki, et al, 2005).

DNA hypomethylation is one of the key events in the initiation of the carcinogenic process in animal models (Jaffe, 2003). DNA hypomethylation largely affects transposons, leading to their activation and promotion of chromosomal rearrangements and other pre-neoplastic changes (Goll and Bestor, 2003). Stable DNA hypomethylation in tissue that undergoes carcinogenesis is also related to cancer progression from normal to tumor cell (Pogribny et al, 2006). Although it is not yet well understood why all cancer tissue does not undergo hypomethylation in the same manner, human cancers can be classified into two groups: a low (0-3.4%) hypomethylation group; and a moderately high (6.8-9.5%) hypomethylation group (Chalitchagorn et al, 2004).

The body of evidence that has accumulated suggests that global hypomethylation may be a potential biomarker for early cancer detection, particularly in populations at risk for cancers lacking effective early detection markers, such as HCC (Giannelli, and Antonaci, 2006; Verma and Srivastava, 2002).

Various chromatin states such as histone modifications (acetylation and methylation) and nucleosome positioning (modulated by ATP-dependent chromatin remodeling machines) determine DNA methylation patterning (Lin et al, 2007). Histone modifications have recently generated a great deal of excitement in epigenetic research, culminating in the histone code hypothesis. The histone code hypothesis predicts that the modification marks on the histone tails should provide binding sites for effector proteins (Stahl and Allis, 2000).

Chromatin, the physiological template of all eukaryotic genetic information, is subject to a diverse array of post-translational modifications that largely impinge on histone amino termini, thereby regulating access to the underlying DNA. The purpose of the chromatin remodeling proteins is to alter the nucleosome architecture such that genes are exposed to or hidden from the transcriptional machinery. The nucleosome can be restructured by two mechanisms: 1. the movement of nucleosomes along DMA which is carried out by ATP-dependent chromatin remodeling complexes; and 2. the modification of core histones by histone acetyltransferases, deacetylases, methyltransferases, and kinases (Cheung et al, 2005).

Distinct histones amino-terminal modifications can generate synergistic or antagonistic interaction affinities for chromatin-associated proteins, which in turn dictate dynamic transitions between transcriptionally active or transcriptionally silent chromatin states. The combinatorial nature of histone amino-terminal modifications thus reveals a “histone code” that considerably extends the information potential of the genetic code. A particular combination of histone tail modifications may be the “code” for the preferential interaction with specific chromatin modifying proteins (Eberharter et al, 2002).

In one embodiment, the present invention provides methods utilizing biomarkers in biospecimens with the intent to detect a change from the normal cell epigenome to a sick cell epigenome, such as an early neoplastic event, a tumor recurrence, or a remnant of oncogenic activity or residual tumor after treatment. In another embodiment, the present invention can also be used as a diagnostic test; for prognostication; to calculate incidence and prevalence rates; and to estimate future burden of disease and associated health care costs.

In one embodiment, the present invention provides a method of detection, including early detection, for cancer, comprising the steps of: isolating DNA from a sample of a subject; and determining a level of global methylation for the DNA, wherein a decrease in the global methylation level as compared to a predetermined normal level indicates the subject has cancer.

In another embodiment, there is provided a method of detection, including early detection, for cancer, comprising the steps of: obtaining a sample from a subject; extracting histone from the sample; determining a global level of histone H4 acetylation and a global level of histone H4 trimethylation, wherein a decrease in the acetylation and trimethylation levels as compared to predetermined normal levels indicates the subject has cancer.

In another embodiment, there is provided a method of detection, including early detection, for cancer, comprising the steps of: isolating DNA from a sample of a subject; extracting histone from the sample; determining a level of global methylation for the DNA; and determining a global level of histone H4 acetylation and a global level of histone H4 trimethylation, wherein a decrease in the global DNA methylation level, and a decrease in the histone H4 acetylation and trimethylation levels as compared to predetermined normal levels indicates the subject has cancer.

The present invention also provides a method of detection, including early detection, for epigenetic changes, comprising the steps of: isolating DNA from a sample of a subject; extracting histone from the sample; determining a level of global methylation for the DNA; and determining a global level of histone H4 acetylation and a global level of histone H4 trimethylation, wherein a decrease in the global DNA methylation level, and a decrease in the histone H4 acetylation and trimethylation levels as compared to predetermined normal levels indicates the subject has epigenetic changes.

### BRIEF DESCRIPTION OF THE DRAWINGS

FIG. 1. Global methylation index in liver cancer cases and controls.

FIG. 2. Graphical expression of the logistic regression, Pr(liver cancer)=logit-1 (b0+b1*methylation) with data overlain. The predictor methylation is the global genomic DNA methylation index value for each case (1) and each control (0).

FIG. 3. Determinants of global DNA hypomethylation and its consequences in a transformed cell (A-C).

FIG. 4. Scatterplot of the global DNA methylation index values for cases and controls with a lowess curve (top row). Distribution histogram of global DNA methylation index values for cases and controls (middle row); Box-Plot of methylation index values in cases and controls (bottom row). The thick line in the middle shows the median, the box shows the 25% and 75% quartiles and the maximum and minimum values for cases and controls are also plotted.

FIG. 5. Distribution plots of predicted methylation index values for 50, 100, 1,000, and 10,000 continuous simulations.

FIG. 6. A. chromatograph of three representative liver cancer samples (A). A chromatograph of non-cancer-controls (B). An elution chromatograph of one single non-tumor tissue (N3083) obtained on four different occasions, after four different protein extractions (C). An elution chromatograph of one single tumor tissue (T2945) obtained on four different occasions, after four different protein extractions (D).

FIG. 7. Scatterplot of modeled simulations (y=□+□X) for HPV infection, smoking and drinking. The methylation index values (the intercept of the glm equation) are plotted on the x axis. Parameter estimates for each covariate are plotted on the y-axis.

FIG. 8. Proposed mechanistic models of epigenetic/genetic alterations in oral and pharyngeal cancer according to etiology: chemical and viral. Smoking (top panel) is associated at the early carcinogenic stage with allelic loss at 3p11, 5q11, 9p21, 17p13, 18q12, gain at 11q13, and amplification of CCND1 gene, loss of p16 and TP53 mutations. HPV16 (bottom panel) initially drives carcinogenesis by inactivating p53 and pRb with the viral oncoproteins E6 and E7, while showing gain at 18q12.

FIG. 9. HPLC chromatogram of DNA digests. The first peak eluting after 3.5+0.5 min corresponded to deoxycytidine, and the second peak eluting after 5.5+0.5 corresponded to 5-methyl 2′ deoxycytidine. A. Representative chromatogram of a liver control sample. B. Representative chromatogram of a tumor liver sample.

FIG. 10. LC-ESI/MS chromatograms for a control sample. A: HPLC separation of deoxycytidine (dC), 5-methyl 2′ deoxycytidine (mdC), deoxyadenosine (dA), deoxyguanosine (dG) and thymidine (dT). Full-scan spectra of dC (B) and mdC (C). ESI conditions were optimized.

FIG. 11. Distribution of methylation index values for cases and controls.

FIG. 12. Box-Plot of methylation index values in cases and controls.

FIG. 13. Scatterplot of modeled simulations (y=α+βx) for HPV, smoking and drinking.

FIG. 14. Environmental determinants of epigenomic regulation at different levels of biological and psychological organization in humans: molecular, cellular, systemic and total body levels.

FIG. 15. Illustrative examples of genomic damage assessment by AP-PCR (left) and DNA methylation changes by AIMS (right). Fingerprints from two matched normal (N)-tumor (T) pairs (patients A and B). Differences of intensity in the tumor with regard to the paired normal tissue (arrowheads) were scored as gains/losses (in AP-PCR) or hypermethylations/hypomethylations (in AIMS).

FIG. 16. Scatterplot of the distribution of the hypomethylation index and the cumulated genomic damage determined by two different techniques in colorectal carcinomas. Box A, GDF detected by AP-PCR in HSP series (n=83). Box B, number of chromosomal alterations detected by CGH in the HUB series (n=50). Boxes C and D, multiple regression analysis after categorization of tumors by the p53 mutational status. p53 mutation showed an additive effect in the number of genetic alterations (GDF, box C; number of chromosomal alterations, box D) to the hypomethylation index. Top regression line, p53 mutated tumors (crosses); bottom regression line, wild-type p53 tumors (open circles).

FIG. 17. Effect of DNA hypomethylation levels on individual chromosome instability. Top, level of hypomethylation in tumors showing alterations in each chromosome or chromosome arm as analyzed by CGH. Points, mean; bars, 95% confidence interval. Chromosomes have been arranged by frequency of alterations (left right). Horizontal dashed line, mean of the hypomethylation level in all tumors. Bottom, distribution of chromosomal alterations in the 50 tumors arranged by the hypomethylation index (down up). Chromosomes have been arranged by frequency of alterations from left (low) to right (high).

FIG. 19. Global DNA methylation values in saliva from premalignant oral cancer patients and controls.

FIG. 20. Risk of bladder cancer in former and current smokers compared with never smokers by methylation quartile

### DETAILED DESCRIPTION OF THE INVENTION

The present invention provides in one embodiment, a method for detecting increased risk of having cancer in a subject, comprising the steps of: (a) isolating a DNA sample from a subject; (b) measuring a value of global DNA methylation index in a sample; and (c) comparing the value of global DNA methylation index in a sample to a standard value of global DNA methylation index (predetermined level), wherein the standard is taken from a cancer-free subject, optionally a disease-free subject or a subject without increased risk of such a cancer or a pool of subjects, whereby if the value of global DNA methylation in the sample is lower than the standard value then the subject has an increased risk of having cancer. The selection and size of an appropriate pool can be readily determined by one of ordinary skill based on well known statistical methodologies and optionally may be selected according to various parameters, including but not limited to one or more of age, gender, health and co-morbid condition.

As used herein, the terms test subject, subject or patient are used interchangeably and refer to a human or another animal species, including primates, rodents (i.e. mice, rats, and hamsters), farm animals, sport animals and pets. In one embodiment, the subject is a human. In certain embodiments, the methods find use in experimental animals, in veterinary application, and/or in the development of animal models for disease.

In another embodiment, the present invention provides a method for detecting increased risk of having or developing cancer in a subject, comprising the steps of: (a) isolating a DNA sample (i.e. as sample comprising DNA) from a subject; (b) measuring a value of global DNA methylation index in said sample; and (c) comparing the value of global DNA methylation index in said sample to a standard value of global DNA methylation index (e.g. a predetermined level), wherein the standard is taken from a subject afflicted with cancer or a pool of subjects afflicted with cancer, whereby if the value of global DNA methylation in the sample is within the standard value and/or limits then the subject has an increased risk of having or developing cancer.

In another embodiment, the present invention provides that a cancerous cell or a pre-cancerous cell has a lower value of global DNA methylation than a standard or normal cell. In another embodiment, the present invention provides that a standard value of global DNA methylation index is a predetermined value derived from healthy or non-diseased cells or a healthy or non-diseased tissue. In another embodiment, the present invention provides that a standard value of global DNA methylation index is a predetermined level derived from experiments assessing a threshold level of methylation in a particular healthy or non-diseased cell type or a particular tissue.

In another embodiment, the present invention provides a method for screening for increased risk of having or developing cancer in a subject, comprising the steps of: (a) isolating a DNA sample from a subject; (b) measuring a value of global DNA methylation index in a sample; and (c) comparing the value of global DNA methylation index in a sample to a standard value of global DNA methylation index, wherein the standard is taken from a healthy subject or a pool of subjects, whereby if the value of global DNA methylation in the sample is lower than the standard value then the subject has an increased risk of having or developing cancer.

In another embodiment, the present invention provides a method for assessing the risk of having cancer in a subject, comprising the steps of: (a) isolating a DNA sample from a subject; (b) measuring a value of global DNA methylation index in a sample; and (c) comparing the value of global DNA methylation index in a sample to a standard value of global DNA methylation index, wherein the standard is taken from a cancer-free subject, optionally disease-free subject or subject without increased risk of cancer or a pool of subjects, whereby if the value of global DNA methylation in the sample is lower than the standard value then the subject has an increased risk of having cancer.

In another embodiment, the present invention provides a method of detection, including early detection, for cancer, comprising the steps of: isolating DNA from a sample of a subject; determining a level of global methylation for the DNA, wherein a decrease in the global methylation level as compared to a predetermined normal level indicates that the subject is afflicted with cancer.

In another embodiment, the present invention provides a method of detection, including early detection, for cancer, comprising the steps of: obtaining a sample from a subject; extracting histone from the sample; determining a global level of histone H4 acetylation and a global level of histone H4 trimethylation, wherein a decrease in the acetylation and trimethylation levels as compared to predetermined normal levels indicate that the subject is afflicted with cancer.

In another embodiment, the present invention provides a method of detection, including early detection, for cancer, comprising the steps of: obtaining a sample from a subject, wherein said sample comprises DNA and histone H4; determining a global level of DNA methylation for said DNA of said sample; determining a global level of histone H4 acetylation for said histone H4 of said sample; and determining a global level of histone H4 trimethylation for said histone H4 of said sample; wherein a decrease in any one or more of the global level of DNA methylation, the global level of histone H4 acetylation, and the global level of histone H4 trimethylation level as compared to normal or standard levels indicates the subject is afflicted with cancer. In one embodiment, a decrease in all of the global level of DNA methylation, the global level of histone H4 acetylation, and the global level of histone H4 trimethylation as compared to normal or standard levels indicates the subject is afflicted with cancer. In certain embodiments, the global level of DNA methylation is determined first. Then, if the results suggest that the subject may be afflicted by cancer or may be at an increased risk of developing cancer, further certainty is sought by testing the global levels of histone H4 acetylation and/or trimethylation. If histone H4 acetylation determination is used as a follow-up study, histone H4 trimethylation can optionally be tested next, or visa versa.

The sample in which the global level of DNA methylation is determined can be the same as or different to the sample in which the global levels of histone H4 acetylation and trimethylation are tested. Similarly, the sample in which the global level of histone H4 acetylation is determined can be the same as or different to the sample in which the global levels of histone H4 trimethylation are tested.

In another embodiment, there is provided a method of detection, including early detection, for abnormal cellular activity, comprising the steps of: isolating DNA from a sample of a subject; extracting histone from the sample; determining a level of global methylation for the DNA; and determining a global level of histone H4 acetylation and a global level of histone H4 trimethylation, wherein a decrease in the global DNA methylation level, and a decrease in the histone H4 acetylation and trimethylation levels as compared to predetermined normal levels indicate the subject has abnormal cellular activity. Methods for the determination of global DNA methylation, global histone H4 acetylation and global histone H4 trimethylation are described herein.

In another embodiment, the present invention provides a method for detecting increased risk of having cancer in a subject, comprising the steps of: (a) isolating a histone sample from a subject; (b) measuring a value of global histone H4 acetylation index and a value of global histone H4 trimethylation index, in the sample; and (c) comparing the value of global histone H4 acetylation index and the value of global of histone H4 trimethylation index in the sample to standard values (predetermined level or values) of global histone H4 acetylation index and global histone H4 trimethylation index, wherein the standard is taken from a cancer-free subject, optionally a disease-free subject or a subject without increased risk of cancer or a pool of subjects, whereby if the value of global histone H4 acetylation index and the value of global histone H4 trimethylation index in said sample is lower than the standard value then the subject has an increased risk of having cancer.

In another embodiment, the present invention provides a method for screening cancer in a subject, comprising the steps of: (a) isolating a histone sample (i.e. a sample comprising histone) from a subject; (b) measuring a value of global histone H4 acetylation index and a value of global histone H4 trimethylation index, in the sample; and (c) comparing the value of global histone H4 acetylation index and the value of global of histone H4 trimethylation index in the sample to standard values of global histone 1-14 acetylation index and global histone H4 trimethylation index, wherein the standard is taken from a cancer-free subject, optionally a disease-free subject or a subject without increased risk of cancer or a pool of subjects, whereby if the value of global histone H4 acetylation index and the value of global histone H4 trimethylation index in said sample are lower than the corresponding standard values then the subject has cancer or has an increased risk of having cancer.

In another embodiment, the present invention provides a method for detecting cancer in a subject, comprising the steps of: (a) isolating a histone sample from a subject; (b) measuring a value of global histone H4 acetylation index and a value of global histone H14 trimethylation index, in the sample; and (c) comparing the value of global histone H4 acetylation index and the value of global of histone H4 trimethylation index in the sample to standard values of global histone H4 acetylation index and global histone H4 trimethylation index, wherein the standard is taken from a healthy subject or a pool of subjects, whereby if the value of global histone H14 acetylation index and the value of global histone H4 trimethylation index in said sample is lower than the standard value then the subject has cancer.

In another embodiment, the present invention provides a method for assessing the risk of developing cancer in a subject, comprising the steps of: (a) isolating a histone sample from a subject; (b) measuring a value of global histone H4 acetylation index and a value of global histone H4 trimethylation index, in the sample; and (c) comparing the value of global histone H4 acetylation index and the value of global of histone H4 trimethylation index in the sample to standard values (predetermined level or values) of global histone H4 acetylation index and global histone H4 trimethylation index, wherein the standard is taken from a cancer-free subject, optionally a disease-free subject or a subject without increased risk of cancer or a pool of subjects, whereby if the value of global histone H4 acetylation index and the value of global histone H4 trimethylation index in said sample is lower than the standard value then the subject has an increased risk of developing cancer.

In another embodiment, the present invention provides a method of detection, including early detection, of an epigenetic change in a subject, comprising the steps of: (a) isolating a DNA sample from a subject; (b) measuring a value of global DNA methylation index in the sample; and (c) comparing said value of global DNA methylation index in the sample to a standard value of global DNA methylation index, wherein the standard is taken from a healthy subject or a pool of subjects, whereby if the value of global DNA methylation index in the sample is lower than the standard value or higher than the standard value then the subject has an epigenetic change.

In another embodiment, the present invention further provides a method for detecting an increased risk of having cancer or detection, including early detection, for cancer in a subject comprising the steps of: (a) isolating a histone sample from a subject; (b) measuring a value of global histone H4 acetylation index in and a value of global histone H4 trimethylation index, in a sample; and (c) comparing the value of global histone H4 acetylation index and the value of global of histone H4 trimethylation index in a sample to a standard values of global histone H4 acetylation index and global histone H4 trimethylation index. In another embodiment, the present invention provides that the standard is taken from a cancer-free subject, optionally a disease-free subject or a subject without increased risk of cancer or a pool of subjects. In another embodiment, the present invention provides that if the value of global histone H4 acetylation index and the value of global histone H4 trimethylation index in a sample are lower than the standard values, then the subject has an increased risk of having cancer.

In another embodiment, a method of detecting or screening an epigenetic change in a subject further comprises the steps of: (a) isolating a histone sample from the subject; (b) measuring a value of global histone H4 acetylation index and a value of global histone H4 trimethylation index, in the sample; and (c) comparing the value of global histone H4 acetylation index and the value of global of histone H4 trimethylation index in the sample to standard values of global histone H4 acetylation index and global histone H4 trimethylation index, wherein the standard is taken from a subject without an epigenetic change, optionally a disease-free subject or a pool of subjects, whereby if the value of global histone H4 acetylation index and the value of global histone H4 trimethylation index in the sample is lower than the standard value or higher than the standard value then the subject has an epigenetic change.

In another embodiment, the methods of the present invention further comprise a method of detecting and/or screening for abnormal stem cell activity by measuring the global levels of DNA methylation, histone H4 acetylation, and/or histone H4 trimethylation. Methods for the determination of the value of global DNA methylation index, value of global histone H4 acetylation index and the value of global histone H4 trimethylation index are described herein.

In another embodiment, there is provided a method of detection, including early detection, for epigenetic changes in brain cells and neurons undergoing Alzheimer's degeneration, comprising the steps of: isolating DNA from a sample of Alzheimer's cases and controls; extracting histone from the sample; determining a level of global methylation for the DNA; and determining a global level of histone H4 acetylation and a global level of histone H4 trimethylation, wherein a decrease in the global DNA methylation level, a decrease in the histone H4 acetylation level, and a decrease in the histone H4 trimethylation level as compared to normal or control levels indicate the subject has a detectable epigenetic change associated with Alzheimer's disease. Methods for the determination of levels of global DNA methylation, global histone H4 acetylation and global histone H4 trimethylation are described herein.

In another embodiment, there is provided a method of detection, including early detection, for epigenetic changes in people with autoimmune diseases like Lupus, comprising the steps of: isolating DNA from a sample of Lupus cases and controls; extracting histone from the sample; determining a level of global methylation for the DNA; and determining a global level of histone H4 acetylation and a global level of histone H4 trimethylation, wherein a decrease in the global DNA methylation level, and a decrease in the histone H4 acetylation and trimethylation levels as compared to predetermined normal levels indicate the subject has a detectable epigenetic change associated with Lupus. Methods for the determination of global DNA methylation, global histone H4 acetylation and global histone H4 trimethylation are described herein. It should be noted that classical autoimmune diseases, such as systemic lupus erythematosus or rheumatoid arthritis, are characterized by massive genomic hypomethylation.

In another embodiment, there is provided a method of characterizing the impact of the environment in modulating the epigenetic markers and gene function by detection, including early detection, of epigenetic changes in people with different environmental exposures and lifestyle associated risk factors for cancer and other complex diseases, comprising the steps of: isolating DNA from a sample, extracting histone from the sample; determining a level of global methylation for the DNA; and determining a global level of histone H4 acetylation and a global level of histone H4 trimethylation, wherein a decrease in the global DNA methylation level, and a decrease in the histone H4 acetylation and trimethylation levels as compared to predetermined normal levels indicate the subject has a detectable epigenetic change associated with different environmental exposures and lifestyle associated risk factors. Determination of global DNA methylation, global histone H4 acetylation and global histone H4 trimethylation are described herein.

In another embodiment, a standard cell is a non-cancerous cell. In another embodiment, a standard cell is a non-cancerous differentiated or non-differentiated cell. In another embodiment, the standard is derived from non-cancerous differentiated or non-differentiated cells. In another embodiment, the standard is derived from a non-cancerous tissue. In another embodiment, the sample and standard are derived from a common tissue but from different sources wherein the standard is derived from a non-cancerous tissue. In another embodiment, the sample and standard are derived from a common tissue but from different sources wherein the standard is derived from a non-cancerous tissue and the sample is suspected of being afflicted with cancer. In another embodiment, the sample and standard are derived from a common tissue and a common source wherein the standard is derived from a non-cancerous cells and the sample is derived from cells suspected of being cancerous cells.

In another embodiment, early detection comprises filtering. In another embodiment, detecting comprises identifying or distinguishing.

In another embodiment, the methods of the invention are based on quantification method of 2′-deoxynucleosides used to evaluate DNA methylation (Fraga, 2005a). In another embodiment, global DNA methylation patterns are obtained using HPLC for fraction separation and Mass Spectrometry for quantification.

In another embodiment, global DNA hypomethylation provides a marker for detection, including early detection, of cancer and other diseases as described herein. In another embodiment, the value of global hypomethylation or a global methylation index or a value of global methylation comprises a quantification method of 2′-deoxynucleosides to evaluate DNA methylation in a diseased cell or a diseased tissue. In another embodiment, determining the value of global DNA hypomethylation or a global DNA methylation index or a value of global DNA methylation comprises a quantification method of 2′-deoxynucleosides to evaluate DNA methylation in a cancerous cell or a cancerous tissue. In another embodiment, determining the value of global DNA hypomethylation level or the value of a global DNA methylation index or the value of global DNA methylation level comprises quantifying 2′-deoxynucleosides to evaluate DNA methylation in cancer cases and controls.

In another embodiment, the level of global DNA methylation is measured and the value of global DNA methylation index is determined by quantifying a global amount of methylated cytosines and a global amount of non-methylated cytosines in the DNA in the sample. In another embodiment, the value of global DNA methylation index is determined by quantifying the global amount of methylated cytosines and the global amount of total cytosines (i.e. methylated and non-methylated) in the DNA in the sample. In another embodiment, the value of global DNA methylation index is determined by quantifying the percentage of methylated cytosine bases in the DNA in the sample calculated from the ratio between methylated cytosine bases in the DNA and the sum of total cytosine bases and methylated cytosine bases in the DNA. In another embodiment, the value of global DNA methylation index is determined by quantifying the percentage of methylated cytosine bases in the DNA in the sample calculated from the ratio between methylated cytosine bases in the DNA divided by the sum of total cytosine bases and methylated cytosine bases in the DNA in the DNA.

For example, the value of global DNA methylation index can be determined by the following formula: (methylated cytosines/(methylated cytosines+total cytosines))×100. In another embodiment, the value of global DNA methylation index can be determined by the following formula: (methylated cytosines/(total cytosines))×100. In yet another embodiment, the value of global DNA methylation index can be the percentage of methylated cytosine bases in the sample calculated from the ratio between methylated cytosine bases in a sample and total cytosine bases in the sample (Fraga, 2005a,b).

In another embodiment, tumor cells comprise aberrant methylation of several CpG islands and global genomic hypomethylation. In another embodiment, tumor cells comprise reduced methylation of several CpG islands. In another embodiment, cancer cells suffer a loss of mono-acetylated and trimethylated forms of histone H4. In another embodiment, loss of mono-acetylated and trimethylated forms of histone H4 appear early and accumulate during the tumorigenic process. In another embodiment, loss of mono-acetylated and trimethylated forms of histone H4 occur predominantly at the acetylated Lys16 and trimethylated Lys20. In another embodiment, loss of mono-acetylated and trimethylated forms of histone H4 are associated with the hypomethylation of repetitive sequences. In another embodiment, the global loss of monoacetylation and trimethylation of histone H4 is a common hallmark of human cancer.

In another embodiment, the epigenetic changes arise long before the neoplasm shows any clinical manifestation of a disease. In another embodiment, the epigenetic changes associated with the earliest stages of neoplastic progression occur before what a pathologist would recognize as a benign pre-neoplastic lesion. In another embodiment, such alterations are inherently polyclonal. In another embodiment, cancer has a fundamentally common basis that is grounded in a polyclonal epigenetic disruption of stem/progenitor cells. In another embodiment, tumor cell heterogeneity is due in part to epigenetic variation in progenitor cells, and epigenetic plasticity, together with gene-environment interaction effects, drives tumor progression. In another embodiment, non-neoplastic, but epigenetically disrupted, stem/progenitor cells in the blood stream or other body compartments are crucial targets for cancer detection, risk assessment and chemoprevention according to the present invention.

The sample is any biological sample or biospecimen comprising DNA and/or histones. In certain embodiments, the histones comprise histone H4. The sample can be a cell sample and/or a tissue sample. Non-limiting examples of suitable samples include blood, saliva, tears, urine, cervical smears, ductal lavage fluids, cerebrospinal fluids, lymph fluids, serosal fluid, bile, stool, tumor biopsies, tissue biopsies, tissue cultures, cell cultures, and primary cell cultures.

The methods of the invention comprise using samples that are biospecimens collected from patients or subject animals; samples that are biospecimens collected from a cell culture, and biospecimens collected from a tissue culture.

In another embodiment, the patient is treated with one or more anticancer agents and monitored according to the methods described herein. In another embodiment, the patient is treated with one or more anticancer agents and monitored before and after treatment according to the methods described herein.

In certain embodiments, the methods described herein comprise detecting cancer or any increased risk of having or developing cancer. The cancer can be any neoplastic disease, including carcinomas, solid tumors and hematologic malignancies. The term “cancer” is also meant to include metastatic disease, metastases, and metastatic lesions, which are groups of cells that have migrated to a site distant relative to the primary tumor. In one embodiment, the cancer is a solid tumor. In another embodiment, the cancer is a hematological tumor. In another embodiment, the cancer is characterized by comprising a metastatic cancer cell population.

In another embodiment, the cancer is liver cancer. In another embodiment, the cancer is oral cancer. In another embodiment, the cancer is prostate cancer. In another embodiment, the cancer is breast cancer. In another embodiment, the cancer is adrenocortical carcinoma, anal cancer, bladder cancer, brain tumor, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, ewings family of tumors (pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, small cell, lymphoma, AIDS-related, lymphoma, central nervous system (primary), lymphoma, cutaneous T-cell, lymphoma, hodgkin's disease, non-hodgkin's disease, malignant mesothelioma, melanoma, merkel cell carcinoma, metasatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cell cancer, salivary gland cancer, sezary syndrome, skin cancer, cutaneous T-cell lymphoma, skin cancer, kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, or wilms' tumor.

The cancer can comprise cancer cells lacking normal Dnmt1 activity. Cancer cells lacking normal Dnmt1 activity can be extensively hypo-methylated in the CpG residues in the 50-end sequences of the 28S and 18S regions of the rRNA gene. Alternatively, or in addition, cancer cells lacking normal Dnmt1 activity can comprise an increase in the acetylation of the lysine 16 residue of histone H4 in the CpG residues in the 50-end sequences of the 28S and 18S regions of the rRNA gene.

In another embodiment, the present methods comprise measuring a loss of trimethylated histone H4-K20 in a sample from a subject, such as a human subject, suspected of having cancer or being at a high risk of having cancer. While not wishing to be bound by theory, it is believed that the loss of trimethylation at histone H4-K20 in DNA repetitive sequences, in conjunction with the loss of DNA methylation, may result in cancer or an elevated risk for cancer. Histone H4 from heterochromatic regions of human cancer cells may also be less trimethylated at K20. Thus, in certain embodiments, the present methods comprise measuring a loss of trimethylated histone H4-K20 in a sample from a subject suspected of having cancer or being at a high risk of having cancer.

In another embodiment, the present invention comprises determining that a cancerous cell or tissue sample or a pre-cancerous cell or tissue sample has lower values of global DNA methylation, lower values of global histone H4 acetylation, and/or lower values of global histone H4 trimethylation than standard values of global DNA methylation, global histone H4 acetylation and/or global histone H4 trimethylation. The standard values of global DNA methylation, global histone H4 acetylation and/or global histone H4 trimethylation may be derived from standard or normal cells or tissue. The standard values can be predetermined values, including pre-determined values derived from healthy cells or healthy tissue. In another embodiment, the present invention provides that standard values of global DNA methylation, global histone H4 acetylation and/or global histone H4 trimethylation are predetermined values derived from experiments assessing a threshold value of global DNA methylation, global histone H4 methylation and/or global histone H4 acetylation in a particular cancer-free, optionally disease-free or healthy cell type or a particular tissue (i.e. a standard cell or tissue).

In another embodiment, a standard cell or tissue is a non-cancerous cell or tissue. In another embodiment, a standard cell or tissue is a non-neoplastic cell or tissue. In another embodiment, a standard cell or tissue is a non-cancerous differentiated or non-differentiated cell or tissue. In another embodiment, the standard is derived from non-cancerous differentiated or non-differentiated cells or tissues. In another embodiment, the sample and standard are derived from a common cell or tissue but from different sources wherein the standard is derived from a non-cancerous tissue. In another embodiment, the sample and standard are derived from a common tissue but from different sources wherein the standard is derived from a non-cancerous tissue and the sample is from a subject having cancer or suspected of being afflicted with cancer. In another embodiment, the sample and standard are derived from a common tissue and a common source wherein the standard is derived from a non-cancerous cells and the sample is derived from cells suspected of being cancerous cells.

In certain embodiments, the cancer is lung cancer and the standard/control is derived from sputum. In other embodiments, the cancer is breast, esophageal, oral, gastric, colon, prostate, liver, kidney, ovarian, cervical, testicular, head and neck and most other solid cancers and the standard/control sample is derived from blood, serum, saliva, and urine among others.

In certain embodiments the DNA is derived from biofluids (such as but not limited to blood, urine, saliva, feces, breast aspirates, semen) and the global level of DNA methylation is measured in the biofluids without the need for measuring a standard or control sample.

In another embodiment, global DNA methylation of a normal cell occurs in the context of other epigenetic marks. In another embodiment, diseases that can be detected or screened for using the present methods have an epigenetic cause. In another embodiment, control of cells by global DNA methylation, global histone modifications, chromatin-remodeling and microRNAs become dramatically distorted in a sick/diseased cell.

A sample can comprise malignant cells having 10-90% less genomic 5 mC than their normal counterpart, such as 10-30%, 30-50%, 50-70%, 60-90%, and/or 30-70% less genomic 5mC than their normal counterpart.

In another embodiment, the global DNA hypomethylation measured in the present methods comprises a loss of methyl groups resulting from hypomethylation of the ‘body’ (coding region and introns) of genes and through demethylation of repetitive DNA sequences, which account for 20-30% of the human genome.

While not wishing to be bound by theory, it is believed that global DNA hypomethylation may contribute to carcinogenesis by any one or more of the following mechanisms: chromosomal instability, reactivation of transposable elements and loss of imprinting. In addition, undermethylation of DNA may favor mitotic recombination, leading to loss of heterozygosity as well as promoting karyotypically detectable rearrangements. Extensive demethylation in centromeric sequences appears to be common in tumors and may induce aneuploidy. Furthermore, the loss of methyl groups may affect imprinted genes and genes from the methylated-X chromosome of women.

In another embodiment, the histone H4 acetylation measured in the present methods comprises Lysine 16 histone H4 acetylation. In another embodiment, the histone H4 trimethylation measured in the present methods comprises Lysine 20 histone trimethylation.

In another embodiment, the value of global histone H4 acetylation index is determined by quantifying the total number non-acetylated, mono-, di-, tri- and tetra-acetylated forms of histone H4. In another embodiment, the value of global histone H4 acetylation index is determined by the following formula: (monoacetylated H4/(monoacetylated H4+diacetylated H4+triacetylated H4+tetraacetylated H4))×100. In another embodiment, the value of global histone H14 acetylation index is determined by quantifying the total number non-acetylated, mono-, di-, tri- and tetra-acetylated forms of histone H4. In another embodiment, the value of global histone H4 acetylation index is determined by the following formula: (monoacetylated H4/(monoacetylated H4+diacetylated H4+triacetylated H4+tetraacetylated H4))×100 (Fraga, 2005a,b).

In another embodiment, the value of global histone H4 acetylation index is the percentage of monoacetylated histone H4 in a sample calculated from ratio between monoacetylated histone H4 in the sample and the sum of monoacetylated histone H4, diacetylated histone H4, triacetylated histone H4, and tetraacetylated histone H14 in the sample. In another embodiment, the value of global histone H4 acetylation index is the percentage of monoacetylated histone H4 in a sample, calculated from the ratio between monoacetylated histone H4 in the sample divided by the sum of monoacetylated histone H4, diacetylated histone H4, triacetylated histone H4, and tetraacetylated histone H4 in the sample.

In another embodiment, the value of global histone H4 trimethylation index is the percentage of trimethylation histone H4 in the sample calculated from the ratio between trimethylated histone H4 index in the sample and the sum of dimethylated histone H4 and trimethylated histone H4 in the sample. In another embodiment, the value of histone H4 trimethylation index is the percentage of trimethylation histone H4 in the sample, calculated from the ratio between trimethylated histone H4 in the sample divided by the sum of dimethylated histone H4 and trimethylated histone H4, in said sample.

In another embodiment, the value of global histone H4 trimethylation index is determined by quantifying the total number dimethylated and trimethylated forms of histone H4. For example, it can be determined by the following formula: (trimethylated H4/(dimethylated H4+trimethylated H4))×100. In another embodiment, histone H4 trimethylation index is determined at Lys20 of histone H4. In another embodiment, the value of global histone H4 trimethylation index is determined by quantifying the total number dimethylated and trimethylated forms of histone H4. For example, the value of global histone H4 trimethylation index is determined by the following formula: (trimethylated H4/(dimethylated H4+trimethylated H4))×100. In another embodiment, histone H4 trimethylation is determined at Lys20 of histone H4. (Fraga, 2005a,b)

In one embodiment, the sample is collected after surgical treatment, radiation therapy, and/or chemotherapy treatment. In another embodiment, the sample is collected before surgical treatment, radiation therapy, and/or chemotherapy treatment. In another embodiment, the sample is collected before and after surgical treatment, radiation therapy, and/or chemotherapy treatment.

In one embodiment, the subject has epigenetic changes related to an autoimmune disease such as, but not limited to, Addison's disease, ankylosing spondilitis, Graves disease, Hashimotos' thyroiditis, Celiac diseases, Chrohn's disease, aplastic anemia, Guillain-Barré syndrome, Kawasaki's disease, rheumatoid arthritis, lupus erythematosus, myasthenia gravis, Sjögren's syndrome, pernicious anemia, multiple sclerosis, or type 1 diabetes mellitus.

In another embodiment, the subject has epigenetic changes related to a neurodegenerative disease.

In another embodiment, the subject has epigenetic changes related to a vascular disease such as, but not limited to, atherosclerosis, peripheral artery disease, aneurysms, renal artery disease, Raynaud's Disease, Buerger's Disease, peripheral venous disease, varicose veins, venous blood clots, deep vein thrombosis, pulmonary embolism, chronic venous insufficiency, blood clotting disorders, or lymphedema,

In another embodiment the subject has epigenetic changes related to a heart disease such as, but not limited to, coronary artery disease, myocardial infarction, angina, acute coronary syndrome, aortic aneurysm and dissections, arrythmias, cardiomyopathy, congenital heart disease, heart failure, peripheral artery disease, or rheumatic heart disease.

In another embodiment, the subject has epigenetic changes related to a metabolic syndrome such as, but not limited to, abdominal obesity, atherogenic dyslipidemia (blood fat disorders—high triglycerides, low HDL cholesterol and high LDL cholesterol—that foster plaque buildups in artery walls), elevated blood pressure, insulin resistance or glucose intolerance, prothrombic state (e.g., high fibrinogen or plasminogen activator inhibitor-1 in the blood), and/or proinflammatory state (e.g. elevated C-reactive protein in the blood stream).

In another embodiment, the subject has epigenetic changes related to an endocrine disorder such as, but not limited to, diabetes mellitus and disorders of carbohydrate metabolism, fluid and electrolyte metabolism disorders, adrenal disorders, thyroid disorders, lipid disorders, acid-base regulation disorders, pituitary disorders, carcinoid tumors, osteoporosis, multiple endocrine neoplasia syndromes, amyloidosis, porphyrias, or polyglandular deficiency syndromes.

In another embodiment, the subject has epigenetic changes related to a behavioral disorder such as, but not limited to, mood disorders, schizophrenia, or bipolar disease.

In another embodiment the subject has epigenetic changes related to a neuromuscular disease such as, but not limited to, amyotrophic lateral sclerosis, or multiple sclerosis.

In another embodiment the subject has epigenetic changes related to a musculoskeletal disease such as, but not limited to, bone diseases, muscle diseases, cartilage diseases, rheumatic diseases, or joint diseases.

In another embodiment the subject has epigenetic changes related to a neurological disorders such as, but not limited to autism, Rett Syndrome, Parkinson's, Ataxia telangiectasia, or Myasthenia gravis.

In another embodiment the subject has epigenetic changes related to an environmental disease, such as, but not limited to, chronic kidney disease associated to exposures to heavy metals or degenerative disease associated to mercury exposure in the diet.

In another embodiment the subject has epigenetic changes related to an occupational disease, such as, but not limited to, asbestosis, adenocarcinoma of the liver or mesothelioma, all diseases with clearly established occupational causal factors.

In another embodiment the subject has epigenetic changes related to an acute or chronic disease as a result of exposure to stressful biopsychosocial causal factors, such as, but not limited to, diseases that define the Status Syndrome, which is linked to the social environment of polluted poor inner city neighborhoods, remote poor rural areas that lack safe drinking water and basic sanitation or marginalized urban sectors that lack social cohesion and have high rates of criminality, abandoned buildings, drug addiction and poverty.

In another embodiment, an epigenetic change in a subject indicates that the subject has an increased risk of being afflicted with an autoimmune disease. In another embodiment, an epigenetic change in a subject indicates that the subject has an increased risk of developing an autoimmune disease. In another embodiment, the autoimmune disease is an organ-specific disease. In another embodiment, the autoimmune disease is a localized autoimmune disease. In another embodiment, an autoimmune disease comprises: Acute disseminated encephalomyelitis (ADEM), Addison's disease, Ankylosing spondylitis, Antiphospholipid antibody syndrome (APS), Aplastic anemia, Autoimmune hepatitis, Autoimmune Oophoritis, Celiac disease, Crohn's disease, Diabetes mellitus type 1, Gestational pemphigoid, Goodpasture's syndrome, Graves' disease, Guillain-Barr syndrome (GBS), Hashimoto's disease, Idiopathic thrombocytopenic purpura, Kawasaki's Disease, Lupus erythematosus, Mixed Connective Tissue Disease, Multiple sclerosis, Myasthenia gravis, Opsoclonus myoclonus syndrome (OMS), Ord's thyroiditis, Pemphigus, Pernicious anaemia, Polyarthritis, Primary biliary cirrhosis, Rheumatoid arthritis, Reiter's syndrome, Sjögren's syndrome, Takayasu's arteritis, Temporal arteritis (“giant cell arteritis”), Warm autoimmune hemolytic anemia, or Wegener's granulomatosis.

In another embodiment, an epigenetic change in a subject indicates that the subject has an increased risk of being afflicted with a neurodegenerative disease. In another embodiment, an epigenetic change in a subject indicates that the subject has an increased risk of developing a neurodegenerative disease. In another embodiment, a neurodegenerative disease comprises: Alexander's disease, Alper's disease, Alzheimer's disease, Amyotrophic lateral sclerosis, Ataxia telangiectasia, Batten disease (Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spielmeyer-Vogt-Sjogren-Batten disease (Batten disease), Spinocerebellar ataxia, Spinal muscular atrophy, Steele-Richardson-Olszewski disease, or Tabes dorsalis.

In another embodiment, an epigenetic change in a subject indicates that the subject has an increased risk of being afflicted with cancer. In another embodiment, an epigenetic change in a subject indicates that the subject has an increased risk of developing cancer.

In another embodiment, the present invention is used as an epigenomic cancer screening and/or detecting tool for early detection of every cancer site/type. In another embodiment, the present invention is used as an epigenomic cancer screening and/or detecting tool of cancer recurrence after treatment of a primary tumor; as a biomarker of therapeutic effectiveness; and as a biomarker of lifestyle and contextual effects related to cancer prevention, diagnosis and progression of disease. In another embodiment, the present invention provides means to decrease mortality rates, increase survival rates and decrease overall cancer associated health care expenditures, by improving detection, including early detection, detection of recurrences, measuring therapeutic effectiveness and monitoring modifiable lifestyle and contextual effects related to cancer.

In another embodiment, the present invention is used as an epigenomic autoimmune disease screening and/or detecting tool for detection, including early detection, of an autoimmune disease. In another embodiment, the present invention is used as a biomarker of therapeutic effectiveness; and as a biomarker of lifestyle and contextual effects related to an autoimmune disease prevention, diagnosis and progression of disease. In another embodiment, the present invention provides means to decrease mortality rates, increase survival rates and decrease overall autoimmune disease associated health care expenditures, by improving early detection, detection of recurrences, measuring therapeutic effectiveness and monitoring modifiable lifestyle and contextual effects related to an autoimmune disease.

In another embodiment, the present invention is used as an epigenomic autoimmune disease screening and/or detecting tool for detection, including early detection, of a neurodegenerative disease. In another embodiment, the present invention is used as a biomarker of therapeutic effectiveness; and as a biomarker of lifestyle and contextual effects related to a neurodegenerative disease prevention, diagnosis and progression of disease. In another embodiment, the present invention provides means to decrease mortality rates, increase survival rates and decrease overall neurodegenerative disease associated health care expenditures, by improving detection, including early detection, detection of recurrences, measuring therapeutic effectiveness and monitoring modifiable lifestyle and contextual effects related to a neurodegenerative disease.

In another embodiment, the present invention is used as an epigenomic schizophrenia screening and/or detecting tool for detection, including early detection, of schizophrenia. In another embodiment, the present invention is used as a biomarker of therapeutic effectiveness; and as a biomarker of lifestyle and contextual effects related to schizophrenia, diagnosis and progression of disease. In another embodiment, the present invention provides means to decrease mortality rates, increase survival rates and decrease overall schizophrenia associated health care expenditures, by improving detection, including early detection, detection of recurrences, measuring therapeutic effectiveness and monitoring modifiable lifestyle and contextual effects related to schizophrenia.

In another embodiment, the present invention is used as an epigenomic bipolar disorder screening and/or detecting tool for detection, including early detection, of schizophrenia. In another embodiment, the present invention is used as a biomarker of therapeutic effectiveness; and as a biomarker of lifestyle and contextual effects related to a bipolar disorder, diagnosis and progression of disease. In another embodiment, the present invention provides means to decrease mortality rates, increase survival rates and decrease overall bipolar disorder associated health care expenditures, by improving detection, including early detection, detection of recurrences, measuring therapeutic effectiveness and monitoring modifiable lifestyle and contextual effects related to a bipolar disorder.

In another embodiment, the present invention is used as an epigenomic bipolar disorder screening and/or detecting tool for detection, including early detection, of diabetes. In another embodiment, the present invention is used as a biomarker of therapeutic effectiveness; and as a biomarker of lifestyle and contextual effects related to diabetes, diagnosis and progression of the disease. In another embodiment, the present invention provides means to decrease mortality rates, increase survival rates and decrease overall diabetes associated health care expenditures, by improving detection, including early detection, detection of recurrences, measuring therapeutic effectiveness and monitoring modifiable lifestyle and contextual effects related to diabetes.

In another embodiment, the present invention is used as an epigenomic bipolar disorder screening and/or detecting tool for detection, including early detection, of ALS. In another embodiment, the present invention is used as a biomarker of therapeutic effectiveness; and as a biomarker of lifestyle and contextual effects related to ALS, diagnosis and progression of the disease. In another embodiment, the present invention provides means to decrease mortality rates, increase survival rates and decrease overall diabetes associated health care expenditures, by improving detection, including early detection, detection of recurrences, measuring therapeutic effectiveness and monitoring modifiable lifestyle and contextual effects related to ALS.

In another embodiment, an epigenetic change in a subject indicates a change in gene expression. In another embodiment, a change in DNA methylation and/or histone methylation and/or acetylation and/or ubiquitylation in a subject indicate a change in gene expression. In another embodiment, an epigenetic change occurs in histone tails are particularly highly modified.

In another embodiment, an epigenetic change in a subject comprises a change in the acetylation of the K14 and/or K9 lysines of the tail of histone H3. In another embodiment, an epigenetic change which includes an acetylation deficiency prohibit transcriptional factors from binding to the DNA, thus the DNA is not exposed to enzymes like RNA polymerase that so transcribe genes. In another embodiment, an epigenetic change which includes an acetylation deficiency inhibits the recruitment of other activating chromatin modifying enzymes. In another embodiment, an epigenetic change which includes an acetylation deficiency inhibits the recruitment of basal transcription machinery.

In another embodiment, an epigenetic change comprises DNA hypomethylation. In another embodiment, an epigenetic change comprises DNA hypomethylation in repeated sequences. In another embodiment, an epigenetic change causes direct increased frequencies of permanent genetic mutation. In another embodiment, an epigenetic change comprises a deficiency in a DNA methyltransferase. In another embodiment, an epigenetic change comprises a deficiency in DNMT1. In another embodiment, an epigenetic change comprises a deficiency in DNMT3A. In another embodiment, an epigenetic change comprises a deficiency in DNMT3B.

In another embodiment, the methods of the invention provide means for identifying compounds that are epigenetic carcinogens provide means for identifying compounds that are epigenetic carcinogens (result in an increased incidence of tumors). In another embodiment, the methods of the invention provide means for identifying compounds that are epigenetic carcinogens but do not comprise mutagen activity. In another embodiment, the methods of the invention provide a global DNA methylation biomarker.

In another embodiment, the global genomic DNA hypomethylation measured in the present methods is a feature of genomic DNA derived from a diseased tissue. In another embodiment, the global genomic DNA hypomethylation is a feature of genomic DNA derived from solid and hematological tumors. In another embodiment, the global genomic DNA hypomethylation is an early epigenetic change from a normal to a diseased cell. In another embodiment, the global genomic DNA hypomethylation is an early epigenetic change from a normal to a pre-malignant cell.

In another embodiment, a global genomic DNA methylation index measuring methylated cytidine relative to global cytidine in the genome is significantly lower in an epigenetic diseased cell or tissue when compared to a control. In another embodiment, a global genomic DNA methylation index measuring methylated cytidine relative to global cytidine in the genome is significantly lower in a cancerous cell or tissue when compared to a control. In another embodiment, global hypomethylation is a very early event in carcinogenesis and can therefore serve as an early indicator of carcinogenesis.

In another embodiment, the present invention also provides a global histone H4 methylation biomarker and/or a global histone H4 acetylation biomarker of detection, including early detection, treatment effectiveness, and relapse monitoring in solid and hematological tumors.

In another embodiment, the present invention further comprises high throughput detection/screening technology in a clinical setting.

In another embodiment, the present invention provides that the methods as described herein are used for staging a tumor. In another embodiment, the present invention provides that global methylation index is a useful biomarker for molecular staging of tumors thus impacting clinical practice and population cancer incidence and prevalence rates.

In another embodiment, the present invention provides that a primary tumor is resected. In another embodiment, the present invention provides that the diseased tissue or tumor undergoes histological examination for evidence of a disease or metastatic potential. In another embodiment, an epigenetic marker of the invention detects early oncogenesis, and thus assists in the detection of clinically silent metastatic disease, before it can be clinically detected as a primary oncogenic lesion, allowing for the treatment of this silent disease, once the organ location is ascertained with the use of a site specific epigenetic index.

In another embodiment, the present invention provides for relapse monitoring of cancer (“sentinel molecular marker”), such as relapse monitoring of breast cancer or colon cancer. In another embodiment, the present methods comprise a sentinel molecular test performed on DNA extracted from blood, saliva, tears, urine, cervical smear, ductal lavage fluid, cerebrospinal fluid, lymph fluid, bile or stool, which could be frequently administered with little discomfort to detect DNA from newly developed cancer cells, and could lead to early detection of recurrences, at an early stage of malignancy, thus increasing survival rates from a secondary tumor. In another embodiment, the present invention provides a method comprising measuring global Histone H4 deacetylation for detection, including early detection, treatment effectiveness, and relapse monitoring in patients having or suspected of having solid and/or hematological tumors.

In another embodiment, the present invention provides methods comprising detecting global epigenetic markers of early cancer progression, such as the loss of DNA methylation, loss of histone H4 acetylation at Lysine 16 and loss of histone G4 methylation at Lysine 20 in liver tissue that are associated with hepatocellular carcinoma (HCC).

In another embodiment, the present invention provides that close relationship between epigenetic modifications of the DNA molecule and large-scale sub cellular phenotypes that define the architecture of a nuclear territory, exist. In another embodiment, the present invention provides that cancer cells lacking Dnmt1 have a substantial disorganization of the nucleolar compartment that is associated with the specific loss of CpG methylation in the rRNA gene repeat.

While not wishing to be bound by theory, it is believed that the loss of DNA methylation measured using the present methods may in some embodiments be due mainly to hypomethylation of repetitive DNA sequences and demethylation of coding regions and introns.

Quantitative methylation assays are known to one of skill in the art. In another embodiment, the present methods comprise a high-throughput quantitative methylation assay that uses fluorescence-based real-time PCR technology. Other suitable quantitative methylation assays comprise bisulphite treatment (in which unmethylated cytosine residues are converted to uracil), and sequence discrimination is achieved by designing the primers to overlap with potential sites of DNA methylation (CpG dinucleotides). In another embodiment, the present invention provides for profiling genome-wide DNA-methylation patterns. In another embodiment, the present invention provides restriction landmark genomic scanning (RLGS). In another embodiment, the present invention provides amplification of intermethylated sites (AIMS) based on arbitrary primed PCR, which does not rely on prior knowledge of sequence information for amplification because. In another embodiment, in AIMS, the DNA templates for amplification are enriched in an initial step that involves digestion with a methylation sensitive restriction enzyme. In another embodiment, the present invention provides methylated DNA immunoprecipitation (methyl-DIP), wherein DNA is first fragmented by sonication and methylated fragments are then immunoprecipitated using a methylation-specific antibody.

In another embodiment, the present invention provides methods for detecting histone modifications. The post-translational histone modifications can be assessed by mass spectrometry. In another embodiment, the present invention provides that global levels of histone modification are obtained by combining other methods. In another embodiment, the present invention provides that histones are isolated by HPLC. In another embodiment, the present invention provides that corresponding eluted fractions are analyzed by HPCE and liquid chromatography-electrospray mass spectrometry (LC-ES/MS). In another embodiment, the present invention provides that modifications at each amino-acid residue are characterized using antibodies in Western blots, immunostaining or tandem mass spectrometry (MS/MS). In another embodiment, the present invention provides the use of ChIP with antibodies against specific histone modifications. In another embodiment, the present invention provides that the immunoprecipitated DNA is analyzed by PCR with specific primers. In another embodiment, the present invention provides the use of ChIP-on-chip with genomic platforms. In another embodiment, the present invention provides extensive maps of histone modifications.

In another embodiment, the present invention provides that global DNA hypomethylation comprises changes in the methylation of CpG rich non-coding areas of the genome such as satellites SAT2 and SAT3, and interspersed repeat sequences such as LINEs, SINEs and long terminal containing repeats (LTRs). It is thought that these changes are evolutionary conserved adaptive responses that maintain homeostasis and assure cell survival in the face of threatening and noxious stimuli.

In another embodiment, the present invention provides that hypomethylation is quantified in a satellite. In another embodiment, the present invention provides that satellite methylation is a surrogate of global hypomethylation. In another embodiment, the present invention provides that hypomethylation is quantified in interspersed repeat sequences, also known as transposable elements.

In another embodiment, the present invention provides that hypomethylation is quantified in retrotransposons. In another embodiment, the present invention provides that hypomethylation is quantified in Human endogenous retroviruses (HERVs) sequences. In another embodiment, the present invention provides that hypomethylation is quantified in HERVs hypomethylation increases with malignancy and is often associated with transcript expression in HERVs hypomethylation is a tumor progression biomarker.

In another embodiment, the present invention provides a genome-wide methylation analysis of non-coding areas of the genome. In another embodiment, the present invention provides comprehensive high-throughput arrays for relative methylation (CHARM) method. In another embodiment, the present invention provides a bio-informatic strategy, averaging information from neighboring genomic locations to obtain highly sensitive and specific statistical result for each subject. In another embodiment, the present invention provides that several samples of genomic DNA from the same subjects are sequenced after having being independently digested and fragmented using assays that enrich for CpGs in different genomic locations by different strategies. In another embodiment, the strategy is MeDIP for CpG islands and gene promoter regions. In another embodiment, the strategy is HELP assay for CpGs in non coding areas of the genome. In another embodiment, the strategy is TIP-Chip assay to enrich for repetitive sequences of the genome.

In another embodiment, the present invention provides adapters that are ligated onto the fragments of genomic DNA that have been processed following three different protocols used for measurement of genome-wide DNA methylation: MeDIP, HELP and TIP-Chip. In another embodiment, the present invention provides a base specific genome-wide map of DNA methylation that characterizes specific tissue/cell/exposure patterns. In another embodiment, the present invention provides a base-specific genome-wide map of histone H4 acetylation and methylation, utilizing a Chip-on-chip protocol to process genomic DNA before ligating it to an oligonucleotide adapters.

Global DNA methylation can be exploited on two additional translational fronts for clinical purposes in cancer patients and for cancer prevention and health promotion purposes in populations. First, using hypermethylation as a complementary gene-specific molecular biomarker of cancer cells to the global methylation marker, because the presence of CpG island hypermethylation of tumor suppressor genes silences them and drives oncogenesis, such as the glutathione S-transferase P1 (GSTP1) genes and others in prostate cancer. (Ellinger, 2008) Hypermethylation could also be used as tool for improving the sensitivity and specificity of global epigenetic biomarkers when detecting cancer cells in multiple biological fluids or even for monitoring hypermethylated promoter loci in serum DNA from cancer patients (Carvalho, 2008, Feng, 2007, Hsiung, 2007). Second, using gene-specific hypomethylation as a molecular biomarker of cancer cells because of the presence CpG island hypomethylation of oncogenes, such as K-RAS in lung cancer (Lockwood, 2008). Third, unlike genetic changes in cancer, epigenetic changes are potentially reversible.

Before the aforementioned CpG islands become hypermethylated, the genome of the cancer cell undergoes global hypomethylation. The malignant cell can have 20-60% less genomic 5mC than its normal counterpart. The loss of methyl groups is accomplished mainly by hypomethylation of the ‘body’ (coding region and introns) of genes and through demethylation of repetitive DNA sequences.

The mechanistic relationships observed in oncogenesis between global hypomethylation and exposure factors have not been established. What is known is that the large decreases in global methylcytosine content can not be due to changes in the methylation of single-copy genes because they account for less than 5% of human DNA.

Global DNA hypomethylation is rather due to changes in the methylation of CpG rich non-coding areas of the genome such as satellites SAT2 and SAT3, and interspersed repeat sequences such as LINEs, SINEs and long terminal containing repeats (LTRs). It is thought that these changes are evolutionary conserved adaptive responses that maintain homeostasis and assure cell survival in the face of threatening and noxious stimuli.

SAT2 and SAT3 satellites, as well as the main representatives of interspersed repeat sequences, LINE-1 (LINEs), Alu (SINES) and human endogenous retrovirus (HERV) sequences, have been investigated in the context of many human cancers. Satellites are normally densely methylated and this contributes to the establishment of heterochromatin. Satellite hypomethylation has been described in several cancers and has been used as a surrogate marker for decreased overall methylcytosine. Satellite methylation is not always a useful surrogate of global hypomethylation because it is not seen in all types of cancers, and even can differ substantially in cancers of the same type.

Interspersed repeat sequences, also known as transposable elements, occupy 45% of the human genome and have the ability to integrate into the genome at a new site within their cell of origin. These elements include (i) DNA transposons, (ii) autonomous retrotransposons, and (iii) non-autonomous retrotransposons. The mechanism that leads to the movement of transposable elements in humans is not well known. Transposable elements move either by a cut-and-paste mechanism (most DNA transposons) or by a copy-and-paste process involving an RNA intermediate (retrotransposons). Significantly, transposable elements related phenotypes do not require disruption of coding sequences.

Defective or evolutionarily divergent elements such as the LINE 1 element in humans can also have profound effects, such as disease susceptibility in response to environmental exposures. DNA transposons are the smallest of the repeat sequences (80-3,000 bp) and probably the oldest element in the human genome. Transposons occupy 3% of the human genome and are mostly degenerate due to internal deletions, end truncations or both, rendering them fixed within the genome. The role of DNA transposons in carcinogenesis and the effects and hypomethylation of transposons in cancer is not well understood.

There are two classes of retrotransposons: those flanked by long terminal repeats (LTRs), largely endogenous retroviruses, and those without LTRs, often referred to as retrotransposons. Human endogenous retroviruses (HERVs) are mostly non-functional LTRs due to the presence of incomplete sequences or mutations. In a limited number of cancers, germ cell tumors and cancers of the ovary, testicles and bladder, HERVs hypomethylation increases with malignancy and is often associated with transcript expression, suggesting a possible tumor progression biomarker role for HERVs in this cancer group. Non LTR retrotransposons are the most prolific repeat sequences in the genome occupying close to 30% of the human genome. Two types have been identified: autonomous Long Interspersed Nuclear Elements (LINEs) and non-autonomous Short Interspersed Nuclear Elements (SINEs).

LINE's range in size between 4-6 kb in length and possess strong internal promoters and encode enzyme that enable integration anywhere in the genome. LINE hypomethylation has been associated to a number of cancers in both tissue and plasma samples. LINE hypomethylation can occur early in cancer of the colon and prostate without a significant correlation to stage. In the other cancers studied, leukemias, urothelial, ovarian and breast cancers, LINE hypomethylation increases with the degree of malignancy, and in some cases has been shown to correlate with clinical outcome. LINE hypomethylation may thus be a useful biomarker for detection, including early detection, and prognostication.

The SINE family of non-autonomous retrotransposons relies on LINEs to enable transposition. The most abundant SINE in humans is the Alu element derived from a 7SL RNA. These elements do not encode proteins but have expanded to cover 11% of the human genome with 1.5 million copies. Analysis of the organization of Alu elements may elucidate the advantage conferred by their non-random genomic distribution, and explain the strong selection in favor of preferential retention of Alu elements in GC-rich regions. Many genes contain 1 or several Alus in close proximity to 5′ of their CpG islands where they may contribute to gene regulation by providing a mark for the edge of the basal promoter. Alus proximate to hypermethylated CpG islands do not usually become hypomethylated in cancer.

The genome uses repetitive elements to protect itself from adverse impacts following exposure to environmental stressors including, but not limited to, oxidant stress, carcinogens, and other deleterious environmental factors. Genome damage has been reported to have an adverse impact on all stages of life including, but not limited to, infertility, fetal development, and accelerated aging, as well as cancer and other degenerative diseases.

The genome is also susceptible to beneficial impacts following exposure to environmental health promoters including but not limited to healthy diets, moderate physical exercise, relaxation techniques, yoga, meditation, massage therapy and other beneficial environmental and external factors. Said factors may counteract some of the deleterious effects resulting from exposure to environmental stressors by reversing epigenetic changes associated to deleterious exposures.

DNA methylation and associated histone modifications are the real “guardian of the genome”, an evolutionarily selected and conserved mechanism that guards the genome from external adverse events and is responsible for genomic maintenance. DNA methylation maintains genome stability by directly stabilizing chromosomes and chromatin compartmentalization, silencing parasitic and viral DNA expression (i.e. LINEs and SINEs), maintaining genomic imprinting and X-chromosomal inactivation, suppressing certain genes for tissue-specific expression, promoting the tissue specific expression of other genes in response to transient and long term mechanisms of adaptation to an ever changing endogenous and exogenous environment at different levels of biological organization: from cell to society.

Global DNA methylation and histone modifications also occupies a place at the crossroads of many pathways in immunology, providing us with a clearer understanding of the molecular network of the immune system. From the classical genetic standpoint, two immunodeficiency syndromes, the ICF (immunodeficiency-centromeric regions instability-facial anomalies) and ATRX (X-linked form of syndromal retardation associated with alpha thalassemia) syndromes, are caused by germline mutations in two epigenetic genes: the DNA methyltransferase DNMT3b and the ATRX genes. Autoimmunity and DNA methylation can also go hand in hand. Classical autoimmune diseases, such as systemic lupus erythematosus or rheumatoid arthritis, are characterized by massive genomic hypomethylation. This phenomenon is highly reminiscent of the global demethylation observed in the DNA of cancer cells compared with their normal-tissue counterparts. Several other examples are also worth mentioning, such as the proposed epigenetic control of the histo-blood group ABO genes and the silencing of human leukocyte antigen (HLA) class I antigens.

Aberrant DNA methylation patterns go beyond the fields of oncology and immunology to touch a wide range of fields of biomedical and scientific knowledge. In neurology and autism research, for example, it was surprising to discover that germline mutations in the methyl-binding protein MeCP2 (a key element in the silencing of gene expression mediated by DNA methylation) causes the common neurodevelopmental disease known as Rett syndrome. This leads us to wonder how many DNA methylation alterations underlie other, more prevalent neurological pathologies, such as schizophrenia or Alzheimer's disease. Beyond that, DNA methylation changes are also known to be involved in cardiovascular disease, the biggest killer in western countries. For example, aberrant CpG island hypermethylation has been described in atherosclerotic lesions. Germline variants and mutations in genes involved in the metabolism of the methyl-group (such as MTHFR) cause changes in DNA methylation, and changes in the levels of methyl-acceptors and methyl-donors are responsible for the pathogenesis of diseases related to homocysteinemia and spina bifida. Imprinting disorders, which represent another huge area of research, are the perfect example of methylation-dependent epigenetic human diseases. A perfectly confined DNA methylation change causes Beckwith-Wiedemann syndrome, Prader-Willi/Angelman syndromes, Russell-Silver syndrome and Albright hereditary osteodystrophy. This highlights the absolute necessity to maintain the correct DNA methylome in order to achieve harmonized development.

Human tumors undergo an overall loss of monoacetylation of lysine 16 and trimethylation of lysine 20 in the tail of histone H4. These two histone modification losses are considered as almost universal epigenetic markers of malignant transformation, as has now been accepted for global DNA hypomethylation and CpG island hypermethylation. Certain histone acetylation and methylation marks may have prognostic value.

The emergence of a new technology for studying DNA methylation based on bisulfite modification coupled with PCR has been decisive in the expansion of the field. The popularization of the bisulfite treatment of DNA (which changes unmethylated ‘C’ to ‘T’ but maintains the methylated ‘C’ as a ‘C’), associated with amplification by specific polymerase chain reaction primers (methylation-specific polymerase chain reaction), Taqman, restriction analysis and genomic sequencing has made it possible for every laboratory and hospital in the world to have a fair opportunity to study DNA methylation, even using pathological material from old archives. We may call this change the ‘universalization of DNA methylation’. The techniques described, which are ideal for studying biological fluids and the detailed DNA methylation patterns of particular tumor suppressor genes, can also be coupled with global genomic approaches for establishing molecular signatures of tumors based on DNA methylation markers, such as CpG island microarrays, Restriction Landmark Genomic Scanning and Amplification of Intermethylated Sites.

The first test of this multiplexed panel is the global methylation test, followed by a histone H4 Lysine 16 acetylation assay. If a sample has negative results for these two markers then the results of the test are deemed negative. Otherwise, a histone H4 Lysine 20 methylation assay is performed. If the results of the three test are positive (low methylation, low acetylation and low trimethylation levels), then the test is deemed to be positive. If the results of these three tests are a combination of positive and negative results, then an algorithm is used to classify the results, taking into consideration the tissue/cell/exposure specific continuous value for each test, the ratios between the values and the different permutations between positive and negative results for these three tests, clinical and demographic characteristics. The algorithm calculates the probability of a positive result. Additional molecular assays may be needed to increase the sensitivity or specificity of the result: Microsatellite analysis; Repetitive elements methylation status assays; Mitochondrial DNA copy number assays; Genome-wide, gene-specific and base-specific methylation and histone modifications assays.

For some conditions and disease states additional molecular tests are needed. These can vary from genome-wide, gene-specific and base specific methylation and histone modification assays; to mitochondrial mutational assays, Single Nucleotide Polymorphisms assays, microRNA expression and target assays, DNA repair activity assays and DNA Methyltransferases activity (DNMT) assays. These assays can be done in samples that give positive and negative results.

The publications cited and/or discussed herein are provided solely for their disclosure prior to the filing date of the present application. Nothing herein is to be construed as an admission that the present disclosure is not entitled to antedate such publication. Further, the dates of publication provided may be different from the actual publication dates, which may need to be independently confirmed. All publications, patents, patent applications and other references cited and/or discussed herein are hereby incorporated by reference.

While the disclosure has been described in detail with reference to certain embodiments thereof, it will be apparent to one skilled in the art that various changes can be made, and equivalents employed, without departing from the scope of the disclosure. In addition, the following examples are illustrative of the methods described herein and should not be considered as limiting the foregoing disclosure in any way.

### EXPERIMENTAL DETAILS SECTION

### Materials and Methods

### Sample Preparation

Frozen tumor tissues from cases and controls were obtained. DNA was extracted using standard methods. Genomic DNA samples were boiled and treated with nuclease P1 (Sigma) for 16 hours at 37° C. and then with alkaline phosphatase (Sigma) for an additional 2 h at 37° C. Global DNA methylation patterns were obtained using HPLC for fraction separation and Mass Spectrometry for quantification. The LC-ESI/MS system consisted of an Agilent Series 1100 HPLC system coupled to an Agilent LC/MSD VL mass spectrometer equipped with an electrospray ionization source (Agilent Technology, Palo Alto, Calif.). 50 μl of the hydrolyzed-DNA solution were injected onto an Atlantis dC18 column (2.1×150 mm; 5 μm particle size) protected by an Agilent guard column (2.1×20 mm; 5 μm particle size) at a constant flow of 0.220 ml min⁻¹. Two buffers, 0.1% formic acid in water (Solvent A) and 0.1% formic acid in 50% water: 50% methanol (Solvent B), were used, with a initial gradient of 5% solvent B, then an increase of solvent B to 50% within 9 min and an isocratic gradient (50% of solvent B) during 25 min.

A drying gas flow of 10 litres per min⁻¹ was employed, with auxiliary 35 psis gas to assist with nebulization and a drying temperature of 350° C. The mass spectrophotometer was operated at a capillary voltage of 4000 V, and spectra were collected in positive ion mode.

### Global DNA Methylation Assay:

Methyl-cytosine concentration in genomic DNA is quantified by Capillary Electrophoresis (CE) techniques. CE has proved to be extremely helpful in separating various DNA components, including a number of bases adducts. The separation and quantification of cytosine and methyl-cytosine were preformed by the use of an SDS micelle system (Fraga, 2004).

### High-Performance Capillary Electrophoresis Quantification of Global H4 Acetylation

H2b, H3 and H4 fractions are separated using High Performance Liquid Chromatography (HPLC) and lyophilized.

Global histone H4 acetylation (AcH4) patterns were quantified as follows: the individual histone H4 fraction is purified by reversed-phase HPLC on a Jupiter C18 column (Phenomenex, Inc.) and eluted with an acetonitrile gradient (20-60%) in 0.3% trifluoroacetic acid using an HPLC gradient system (Beckman Coulter). Non-, mono-, di-, tri- and tetra-acetylated histone H4 derivatives were resolved by HPCE. An uncoated fused-silica capillary (Beckman-Coulter; 60.2 cm×75 μm, effective length 50 cm) was used in a capillary electrophoresis system (P/ACE MDQ, Beckman-Coulter) with 100 mM phosphate buffer (pH 2.0) 0.02% (w/v) HPM-cellulose as running buffer and operating voltages of 12 kV. Data was collected from four separate experiments. Three measurements were made per sample (Boix-Chornet, 2006, Fraga, 2005).
Identification of 2′-deoxycytidine

Identification of 2′-deoxycytidine (dC) and 5-methyl-2′-deoxycytidine

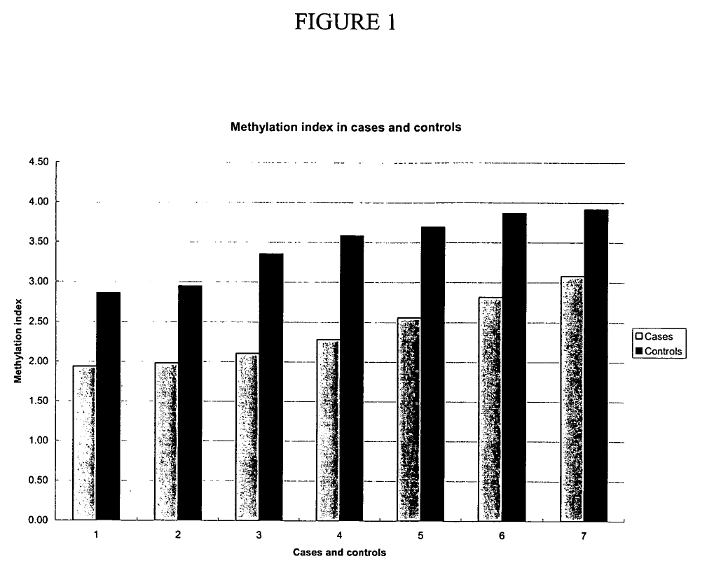

(5 mdC) was obtained by UV detection at A₂₅₄ and A₂₈₀. Quantification of global DNA methylation was obtained from integration peak areas of 5 mdC relative to global cytidine (5 mdC+dC). FIG. 1 shows a representative chromatogram of a liver control sample (A) and a tumor liver sample (B).

### Methylation Index Validation

Methylation index was validated in the different phases of biomarker development for different types of tissues phenotypes (Brena, 2006, Fraga, 2005, Frigola, 2005, Paz, 2004). An immunohistochemical biomarker test is developed and calibrated against the methylation index of each cancer site created with the HPLC/Mass Spectrometer (Honrado, 2007). An immunohistochemical test is used for clinical analysis using automated readers.

### DNA-Protein Interactions

### Statistical Analysis and Hypothesis Testing

Descriptive statistics showing univarible, bi-variable and multivariable characteristics are performed utilizing STATA version 9.0 (Stata Corporation, Texas, 2006). Hypothesis testing is preceded by descriptive statistics, including examination of frequency distributions and measures of central tendency and variation, simple correlations among variables, and their variance-covariance matrix. Values were log-transformed as needed to normalize the distributions. The main hypotheses tests (e.g. paired t tests) use p<0.05 and a 2-tailed test unless otherwise stated. Due to the pilot nature of this proposal we have not conducted power calculations.

Variables of interest are dichotomous Binding fit for histone H4 and global DNA methylation index as major exposure factors of HCC and as outcome variables in different hierarchical logistic regression analyses controlling for HCC risk factors. A Bayesian hierarchical glm package for R version 2.4.1 is used to perform hierarchical case-control analyses, using epigenetic patterns as both, exposure factor and outcome variable.

### Bisulfite Conversion of DNA and Validations by COBRA and Cloning/Sequencing

The methylation data for selected SNPs were validated by bisulfite conversion using the CPGENOME DNA Modification Kit (Chemicon) followed by PCR amplification and either restriction analysis (COBRA) or cloning and sequencing. PCR was done using PLATINUMTAQ (Invitrogen) and with locus-specific primers matching the bisulfite converted sequences flanking the CpG dinucleotides to be assayed. PCR primers were selected using METHPRIMER, primer sequences are available on request, and COBRA restriction enzymes were selected using SNAKE-CHARMER. Bisulfite converted/PCR amplified DNA from pre- and post-treatment bone marrow aspirates are cloned using the TOPO-TA Cloning Kit (Invitrogen) and multiple clones are sequenced.

### Example 1

### Epigenetic Biomarkers for Liver Carcinoma

Global hypomethylation is a very early event in human and experimental hepatocarcinogenesis and a feature of genomic DNA derived from solid and hematologic tumors, which may precede region-specific hypermethylation in neoplastic transformation from normal to pre-malignant phenotypes (Shen et al, 1998). Global hypomethylation in serum has been proposed as a potential prognostic marker for hepatocellular carcinoma (Tangkijvanich, 2007).

Aberrant methylation of four genes, COL1A2, IGFBP2, CTGF and fibronectin 1, has been detected in both hepatoma cell lines and primary hepatoma tissues. In addition, methylation of 5′CpG islands and histones deacetylation coexisted in the regulation of gene expression of COL1A2, IGFBP2, CTGF, but not of fibronectin 1, suggesting that both DNA methylation and histones deacetylation, occur in patterns closely associated with altered gene expression in hepatoma (Tada et al, 2005).

Hypermethylation of tumor suppressor genes, the best understood methylation marker in cancer, has also been documented in Hepatocellular carcinoma (HCC) (Yu et al, 2002; Yu et al, 2003; Herath et al, 2006; Lee et al, 2003; Fang et al, 2003). Hypermethylation of over 100 tumor suppressor genes in HCC has been shown to be divided into four different categories of genes, which inhibit three different cell signaling pathways (Ras, Jak/Stat, Wnt/-catenin) and increases during HCC development and progression (Calvisi et al, 2007). Etiology independent oncogene activation has also been documented to play an important role in HCC (Schlaeger et al, 2008).

Frozen tissue samples from liver cancer patients and controls were obtained from the Cooperative Human Tissue Network.

DNA was extracted using standard methods. Five micrograms of genomic DNA samples were boiled and treated, with nuclease PI and alkaline phosphatase.

Global genomic DNA methylation patterns were obtained using HPLC for fraction separation and Mass Spectrometry for quantification.

Fifty micrograms of the hydrolyzed-DNA solution were injected onto a reversed phased Atlantis dC 18 column (2.1×150 mm; 5 μm particle size) protected by an Agilent guard column (2.1×20 mm; 5 μm particle size) at a constant flow of 0.220 ml/min. Two buffers, 0.1% formic acid in water (Solvent A) and 0.1% formic acid in 50% water: 50% methanol (Solvent B), were used, with an initial gradient of 5% solvent B, then an increase of solvent B to 50% within nine minutes and an isocratic gradient (50% of solvent B) during 25 minutes. Identification of 2′-deoxycytidine (dC) and 5-methyl-2′-deoxycytidine (5 mdC) was obtained by UV detection at A₂₅₄ and A₂₈₀₄ using a LC-ESI/MS system. Quantification of global genomic DNA methylation was obtained from integration peak areas of 5 mdC relative to global cytidine (5 mdC+dC). The DNA methylation index was obtained in triplicate for each sample. The LC-ESI/MS system consisted, of an Agilent Series 1100 HPLC system coupled to an Agilent LC/MSD VL mass spectrometer equipped with an electrospray ionization source (Agilent Technology, Palo Alto, Calif.).

A drying gas flow of 10.0 ml/min. was employed, with auxiliary 35 psi gas to assist with nebulization and a drying temperature of 350° C. The mass spectrophotometer was operated at a capillary voltage of 4,000 V and spectra were collected in positive ion mode.

Significance of results was ascertained with a two-sample t-test using Welch's approximation for samples with unequal variances and a Wilcoxon rank sum test. Significance analyses were conducted in Stata 9.0 (Stata Corporation, Texas, 2006). Logistic regression modeling and graphical representation were done in R 2.5 (R-Project, 2007).

### Results

The results of this study are shown in Table 1. The median (range) of the global genomic DNA methylation index value was 2.42 (1.94-3.08) for cases and 3.64 (2.86-4.13) for controls. The standard deviation for the global genomic DNA methylation index was 0.42 for cases and 0.46 for controls and the interquartile range was 1.14 for cases and 1.27 for controls (Guerrero-Preston, 2007).

TABLE 1
DNA samples in triplicate (n = 10 pairs)

| | Cases | Controls |
|---|---|---|
| Methylation index median [range] | 2.42 (1.94-3.08) | 3.64 (2.86-4.13) |
| Methylation mean (95% CI) | 2.43 (2.08-2.78) | 3.55 (3.16-3.93) |
| Standard Deviation | 0.42 | 0.46 |
| Interquartile Range | 1.14 | 1.27 |

The mean global genomic DNA methylation index value, measuring methylated cytidine relative to global cytidine in the genome, was significantly lower (p value=0.001 for two sample t-test; p value=0.01 for Wilcoxon rank-sum test) for all cases, mean=2.43 (95% CI, 2.08, 2.78), when compared to controls, mean=3.55 (95% CI, 3.16, 3.93). The significant difference in means and the lack of overlap in confidence intervals for cases and controls suggest that the global genomic DNA methylation index is a useful epigenetic biomarker to distinguish between liver cancer cases and controls.

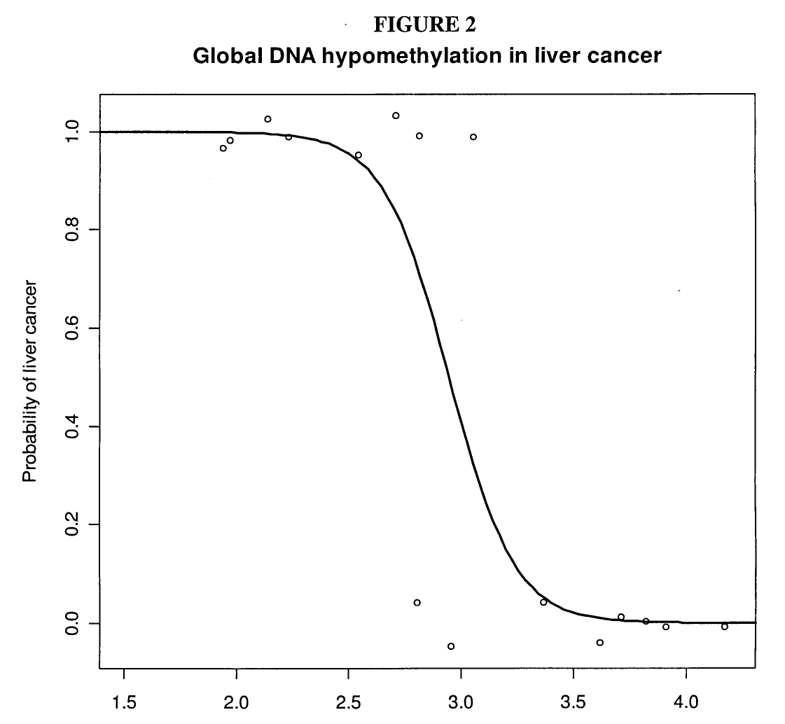

FIG. 2 shows a graphical expression of the logistic regression described by the following expression: Pr (liver cancer)=logit⁻¹ (β₀+β_{i,j}*methylation) with data overlain. The predictor methylation is the global genomic DNA methylation index value for each case (1) and each control (0).

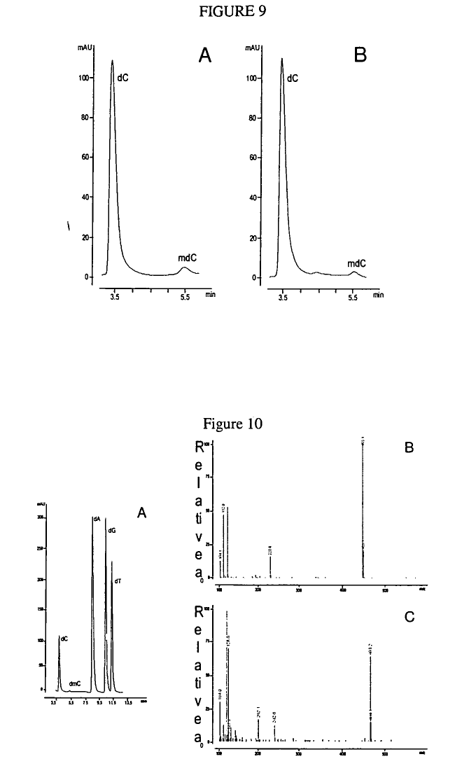

The Chromatograms in FIG. 9 show a representative plot of a case and a control. The Chromatogram and full scan spectra in FIG. 10 show the results for a control sample.

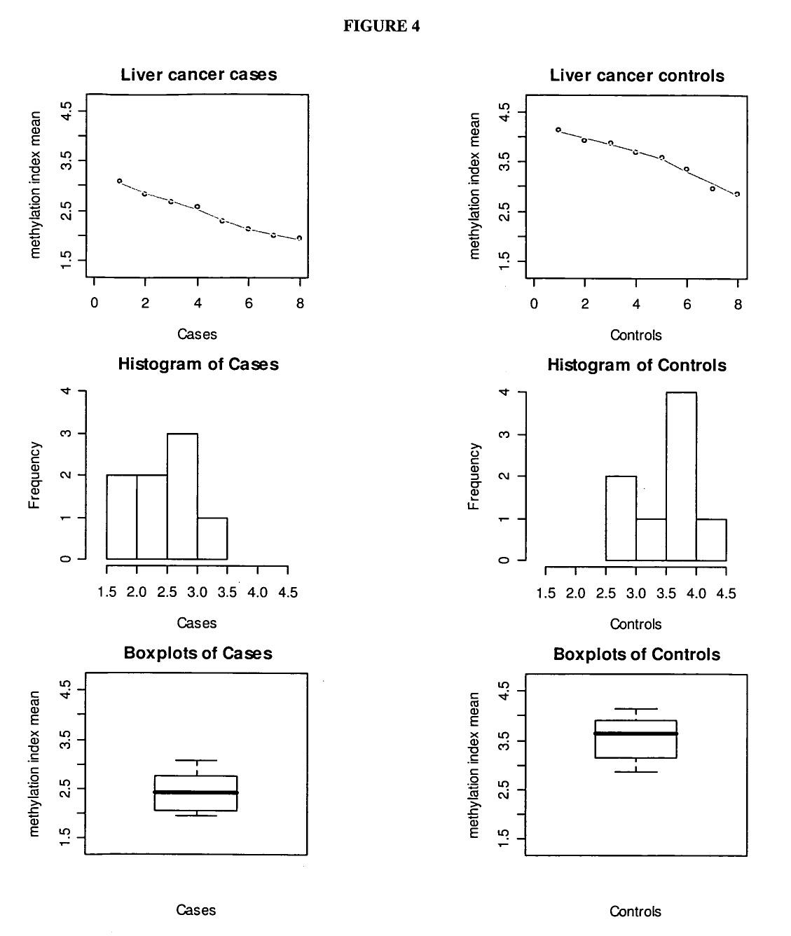

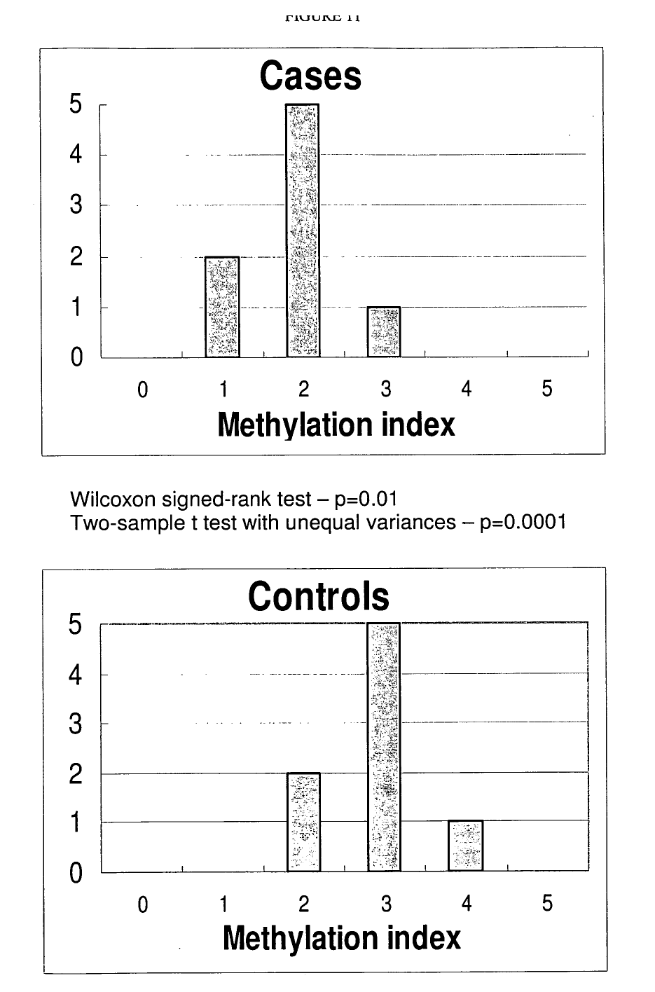

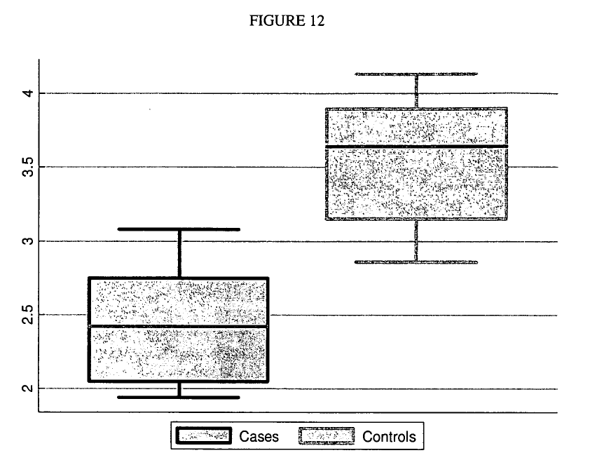

The mean results of the DNA methylation index for cases and controls are plotted in decreasing order. The distribution of methylation index values for cases and controls appear to follow a symmetric distribution in the histograms (FIG. 4). The distribution plots in FIG. 11 show histograms for cases and controls. The Box Plot in FIGS. 11 and 12 shows the median, 25% and 75% quartiles and the maximum and minimum values for cases and controls. Regardless of how the data is visualized, the methylation index distinguishes cases from controls.

### Simulations

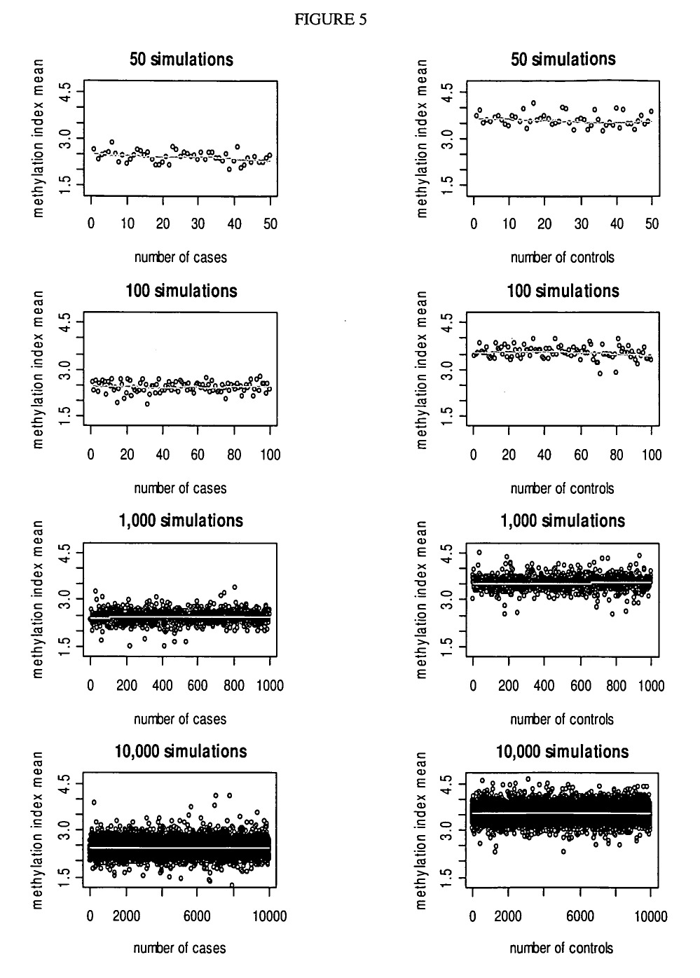

Results from this proof-of-principle experiment were used to simulate the predicted range of methylation index values for human liver cancer cases and controls in which future methylation index values are expected to be found. Continuous predictive simulations were performed to create a methylation index probability distribution curve using a Bayesian method of weighted data averaging for sparse data. A continuous predictive distribution of the global DNA methylation index was obtained in R for 50, 100, 1,000, and 10,000 simulations of liver cancer cases and controls. The results are presented in FIG. 5. These distributions represent the values of the posterior distribution of the mean of the methylation index, one of the unknown hyperparameters in a hierarchical Bayesian model that can be used to predict future methylation index values based on the observed values obtained in this experiment. [22] Briefly, the probability of a predicted methylation index (θ) based on the observed methylation index mean (γ) and the unknown hyperparameters (μ) and (τ) is given by the following model,

θⱼ|μ,τ,γ˜N(θ·hat,Vⱼ),

where θ·hat is a precision weighted average of the prior population mean (μ) and the sample mean (γ), given a prior precision (τ). The prior population mean μ can be thought of as the distribution of all possible methylation index values from where the actual data were obtained in this experiment. We combine the data with the uniform prior density p(μ|τ) to obtain the posterior density for μ, given τ using the following model,

μ|τ,γ˜N(μ·hat,V_{μ})

The mean and predicted 95% confidence intervals for cases 2.43 (2.05, 2.93) and controls 3.54 (3.11, 4.01) do not overlap in the simulations. These simulated results provide a range of values and confidence intervals within which we expect to find the majority of methylation index values for all human liver cancer cases and controls.

Global genomic DNA methylation index was successful in distinguishing between tissue samples of liver cancer cases and controls. Liver cancer was used as proof-of-principle study because most of the animal work in carcinogenesis has been done in hepatocarcinogenesis.

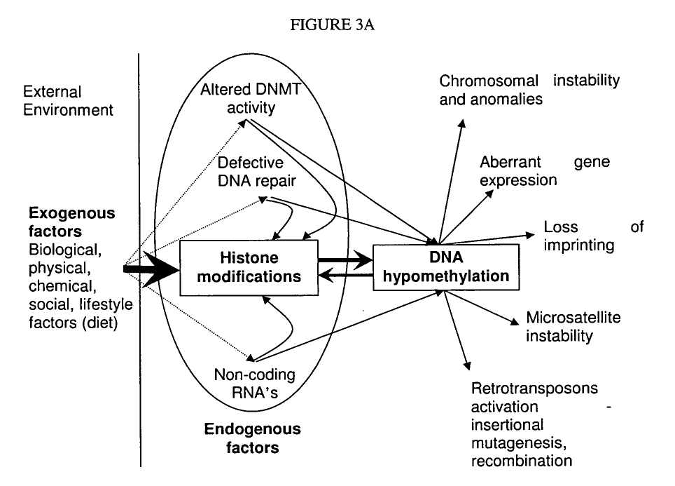

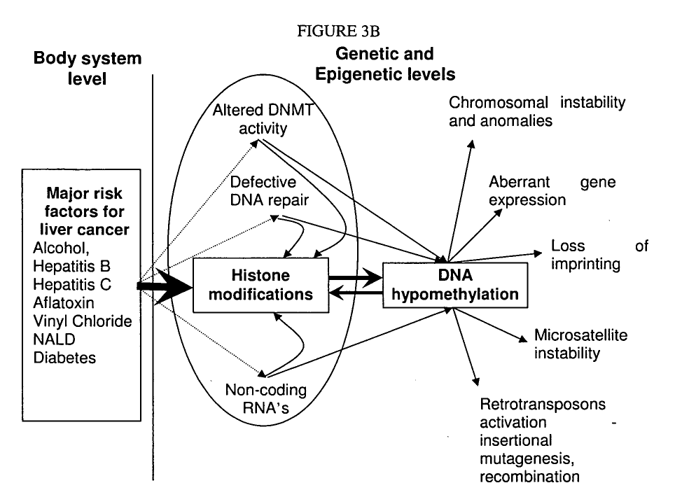

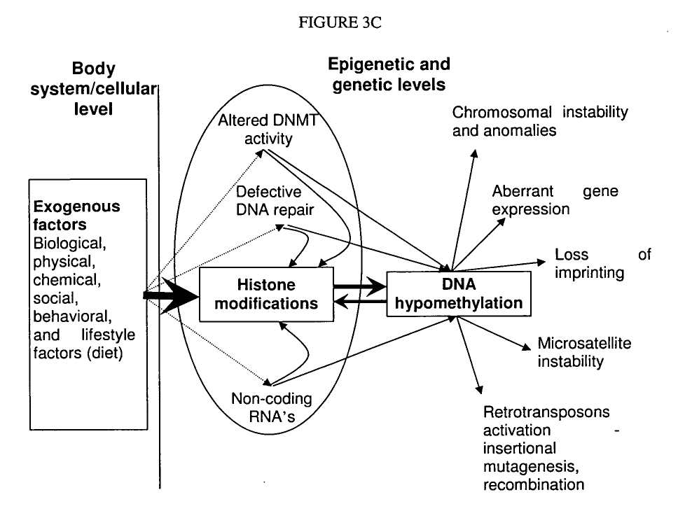

The advantages of a global genomic DNA methylation index as an detection, including early detection, tool in human cancer is further described in the conceptual model of the determinants of DNA hypomethylation (FIG. 3).

In the main pathway proposed (FIG. 3), exogenous factors (biological, chemical, physical, social and life-style factors) cause histories modifications that lead to global DNA hypomethylation (Fraga, 2005b). Endogenous factors acting through three secondary pathways related to decreased DNA methyltransferase expression, (Dodge et al, 2005; James et al, 2006) non-coding RNA silencing (Lujambio et al, 2007; Costa, 2005) and defective DNA repair, (Koturbash, 2005; Pogribny et al, 2005) have a direct causal role in global DNA hypomethylation. Factors that activate these three endogenous pathways can lead directly to a loss of global. DNA methylation may also cause chromatin modifications leading to hypomethylation, or may only be an intermediate step leading to histones modifications that are linked to global DNA hypomethylation.

The model also shows how the global loss of methylation, mediated by the interaction of exogenous and endogenous factors, leads to abnormalities associated with pre-malignancy and malignancy: chromosomal instability, aberrant gene expression, loss of imprinting, micro satellite instability and retrotransposons activation.

The two-sample t-test and the Wilcoxon rank sum test showed a significant difference between the global genomic DNA methylation index in cases and controls. The enzymatic hydrolysis method and the LC-ESI/MS assay utilized allows for the quantification of total methylated cytosines in the genome and the calculation of a relative methylation index, which is used to effectively compare methylation changes across different tissues.

These results clearly demonstrate that liver cancer cases can be distinguished successfully from controls distinguish using a global genomic DNA methylation assay. Since global DNA hypomethylation is tissue specific in cancer, a continuous global genomic DNA methylation index is a useful early epigenetic biomarker for cancer research.

Once the global genomic DNA methylation index of liver cancer cases and controls is validated in blood samples, correlation studies are performed in a prospective cohort to compare the sensitivity and specificity of global DNA hypomethylation as an detection, including early detection, marker of liver cancer against the current diagnostic marker in blood, circulating levels of α-fetoprotein.

In conclusion, a correlation between global DNA methylation patterns and liver tissue was observed utilizing an innovative enzymatic hydrolysis quantification method. These results suggest that global DNA hypomethylation is a useful epigenetic biomarker for detection, including early detection, of cancer progression in high risk individuals or as a biometric of adjuvant and neoadjuvant treatment effectiveness in hepatocellular carcinoma. The data derived from analysis of cancer cells, suggest a potential association between loss of cell-growth control and altered differentiation with global hypomethylation, mainly due to loss of methylated cytosines in repetitive sequences throughout the genome. Moreover, hypomethylation of repetitive elements in cancer is directly linked to the neoplastic process and not a simple consequence of loss of growth control observed in most of the cancer cells. The knowledge obtained from this project may lead to the development of epigenetic biomarkers for early detection (Winget et al, 2003) relapse monitoring (Nomoto et al, 2007), and secondary HCC prevention (Verma and Srivastava, 2003).

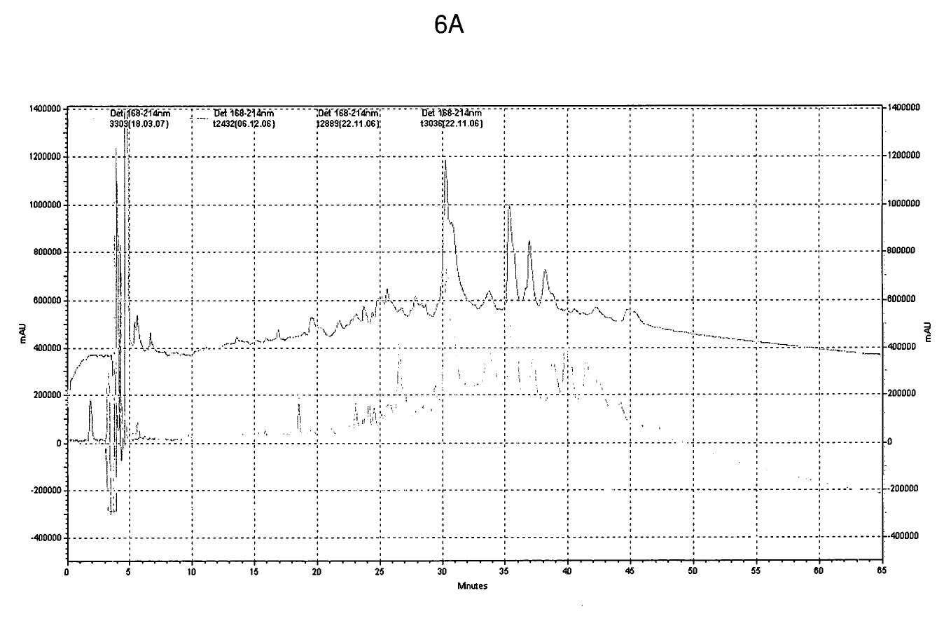

Global histone H3 and H4 fractions were isolated and individual histone H3 and H4 fractions from liver tissue samples were purified by reversed-phase HPLC on a Jupiter C18 column (Phenomenex, Inc.) and eluted with an acetonitrile gradient (20-60%) in 0.3% trifluoroacetic acid using an HPLC gradient system (Beckman Coulter). FIG. 6A shows the chromatograph of three representative liver cancer samples. The three peaks that elute between minutes 35 and 40 are a characteristic of cancer tissues and are not observed in non-cancer controls, as shown in FIG. 6B. FIG. 6C shows the elution chromatographs of one single non-tumor tissue (N3083) obtained on four different occasions, after four different protein extractions. FIG. 6D shows the elution chromatographs of one single tumor tissue (T2945) obtained on four different occasions, after four different protein extractions.

These series of chromatographs clearly demonstrate that liver cancer cases can be distinguished successfully from controls by simply looking at the elution patterns of histone H3 and H4 fractions when they are fractionated from other tissue proteins.

A similar distribution of Histone H3 and H4 peaks is seen in tissues from oral cavity cancer patients, but in this case among the cases, which is probably a reflection of the tissue specificity of these biomarkers.

### Example 2

### Global DNA Methylation: a Common Early Event in Oral Cancer Cases with Exposures to Environmental Carcinogens or Viral Agents

A proof-of-principle study was performed to ascertain if global DNA methylation could be a useful tool in distinguishing early molecular changes in OCP.

### Method

Tissue samples from fifteen oral cavity cancer cases were collected from surgical specimens of HNSCC tissue banked at the Tumor Biology Laboratory of the University Of Puerto Rico School Of Medicine for this proof-of-principle study. Personal histories of tobacco and alcohol use were ascertained by questionnaire. HPV infection was determined by detecting HPV DNA in tumor tissue by polymerase chain reaction (PCR). DNA was extracted using standard methods. Genomic DNA samples were boiled and treated with nuclease P1 and alkaline phosphatase. Global DNA methylation levels were obtained using HPLC for fraction separation and Mass Spectrometry for quantification. 50 μl of the hydrolyzed-DNA solution were injected onto a reversed phase dC18 column. Two buffers, 0.1% formic acid in water and 0.1% formic acid in 50% water/50% methanol, were used.

Identification of 2′-deoxycytidine (dC) and 5-methyl-2′-deoxycytidine (5 mdC) was done by UV detection at A254 and A280. Quantification of global DNA methylation was obtained from integration peak areas of 5 mdC relative to global cytidine (5 mdC+dC). Significance of associations between the methylation index and predictor variables, age, gender, smoking, alcohol and hpv insertion was ascertained in Stata 9.0 with a bivariable Pearson's Chi squared test. Predictive simulations were performed to explore associations between etiological factors and global DNA methylation. Generalized linear models were fitted, predictive simulations were implemented and scatterplots were made in R 2.6. We can use the sim( ) function in R to create simulations that represent our uncertainty in the estimated regression coefficients.

### Results

The global methylation index, measuring methylated cytosine over total cytosine in the genome, was found to be 4.28 (95% CI, 4.1, 4.4) in an oral cancer case series. The Pearson's chi squared test showed no statistically significant differences in the association between the global DNA methylation levels of cases that had smoking (p=0.21), drinking (p=0.31) or HPV insertion (p=0.34) as etiologic factors, when compared to cases that did not.

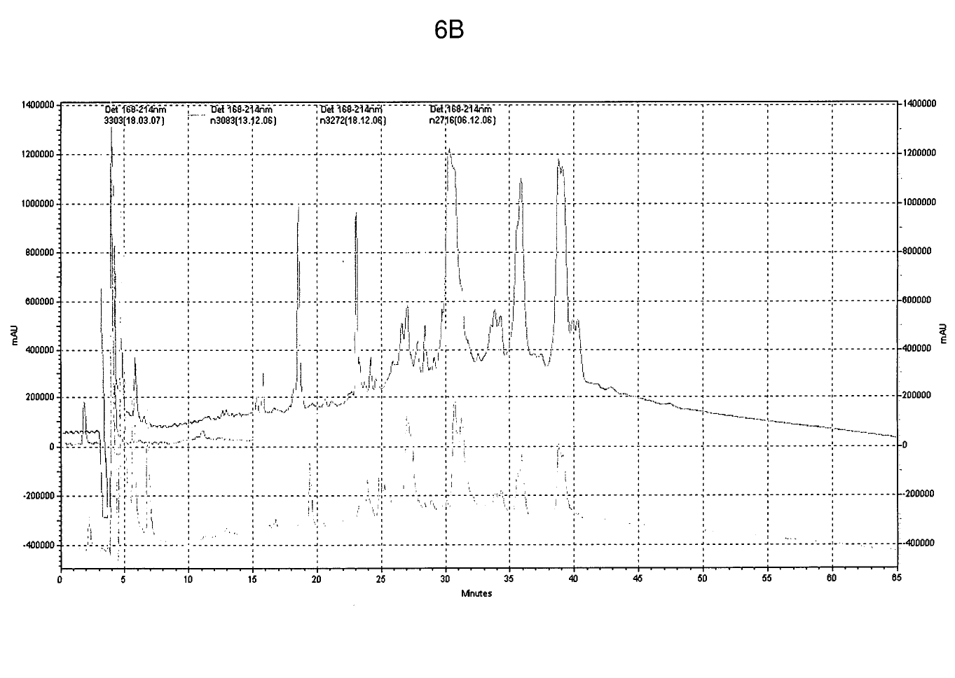

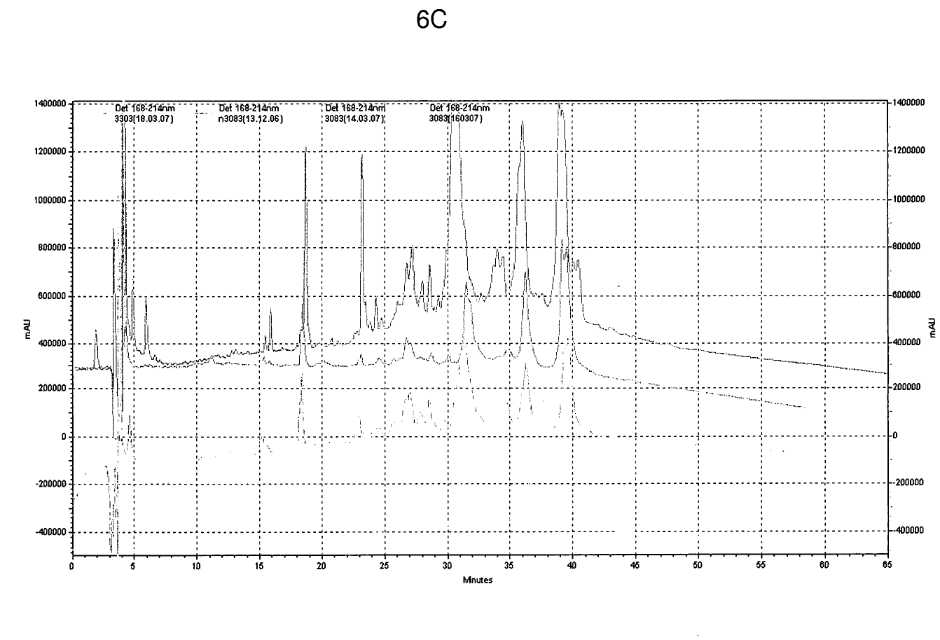

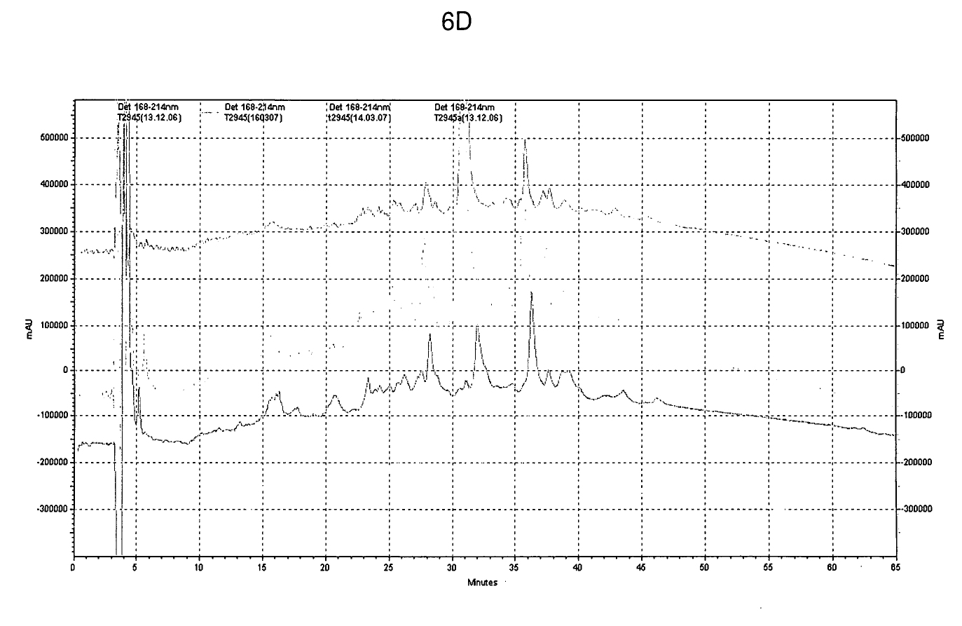

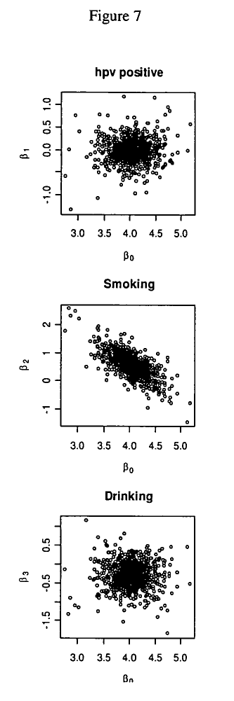

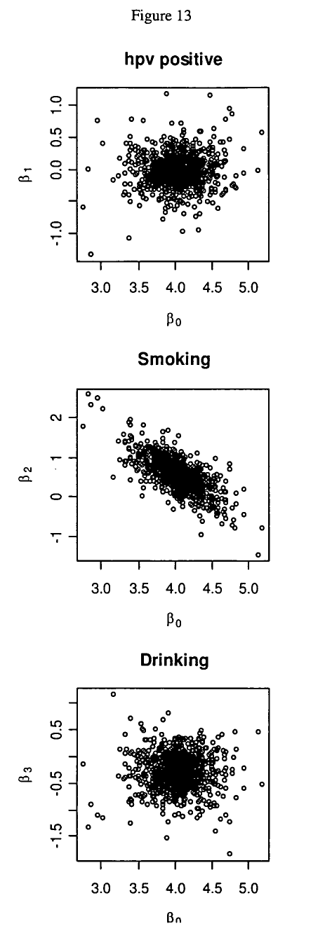

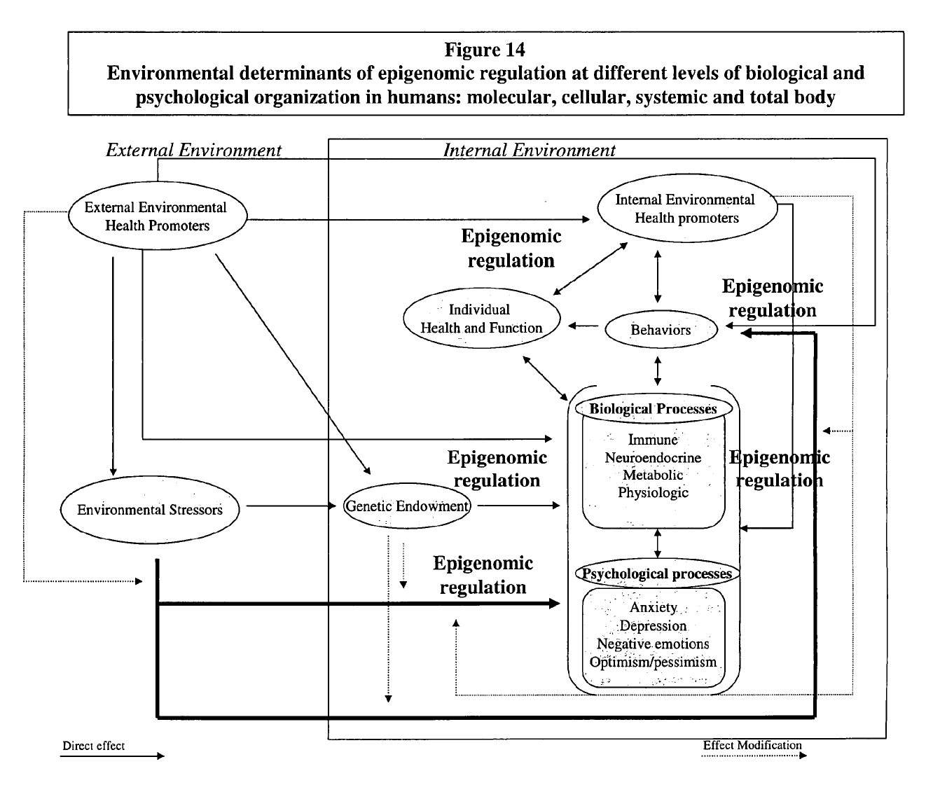

An inverse association between smoking and DNA methylation was observed (FIG. 13) after 1,000 simulations of the glm linear model (y=α+βX). As the probability of smoking increases the probability of DNA methylation decreases (FIG. 7). No associations were observed between the probability of DNA methylation and drinking or HPV infection after 1,000 simulations.

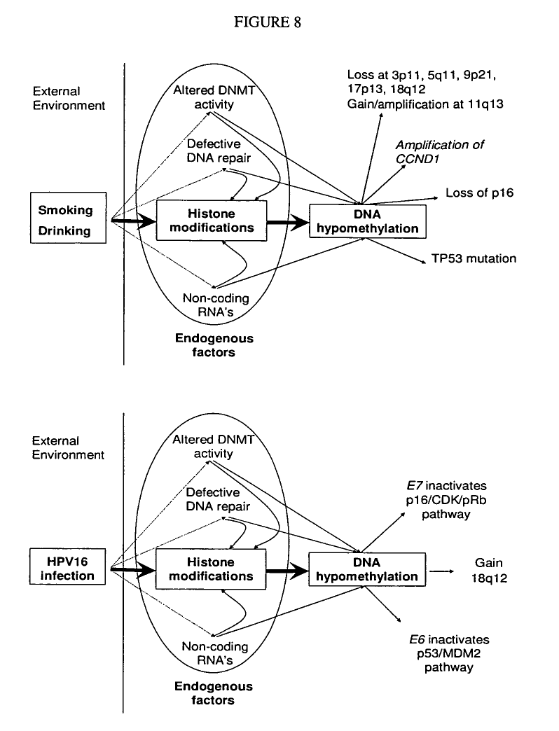

Tissue specific global methylation was shown for oral cancer cases with different etiologies, with a mean and standard deviation different than those previously found by us in liver cancer tissue using the same methodology. No difference in global DNA methylation levels between cases with different etiologies was observed, although smoking was correlated to DNA methylation levels when continuous predictive simulations utilizing a generalized linear model were performed. These preliminary in-vivo and in-silico results suggest that global DNA methylation may precede genetic alterations and molecular changes associated with exposure to viral and environmental carcinogens in HNSCC, as our conceptual model depicts (FIG. 8). Many methylated cytosines have been found in retrotransposons, endogenous retroviruses and repetitive sequences, which may have evolved as a host defense mechanism to prevent the mobilization of these parasitic elements and reduce the occurrence of chromosomal rearrangements and the gain or loss of whole chromosomes (aneuploidy). [19-21]

Aneuploidy may be observed during chromosomal instability. DNA methylation has been associated with such instability. Loss of genomic integrity has been attributed to hypomethylation of repetitive elements, which can lead to inappropriate recombination resulting in defects in cell cycle monitoring check point genes as well as genes involved chromosome condensation, kinetochore structure and function, and centrosome and kinetochore formation. Chromosomal breakage and translocations in rare recessive genetic disorders are suggested to be due to mutations in the methyltransferase gene DNMT3b. Hypomethylation-induced translocations have been observed in multiple myeloma. DNA methylation seems to be a stabilizing agent in genomic structures comprising large amounts of repetitive elements by preventing recombination across these regions.

Global genomic DNA hypomethylation precedes and subsequently coexist with gene-specific promoter hypermethylation and hypomethylation in cancer. Gene-specific hypermethylation has been associated with silencing of tumor suppressor genes. Gene specific hypomethylation has been associated with activation of oncogenes. The relationship between hypomethylation and hypermethylation in cancer is not well understood.

Screening high risk populations for oral cancer in the primary care setting has been shown to be effective. Nevertheless, a systematic review of existing early detection programs have not shown to be effective in impacting the burden of disease. A global DNA hypomethylation index, capturing loss of methylation at interspersed repeat sequences and genes, is a valid biomarker for the early detection of tumors and for prognostic use in monitoring disease progression. The sensitivity and specificity of this marker may be improved if it is combined with global histones H4 modification markers.

A surveillance program measuring global epigenetic biomarkers for OCP in saliva in high risk populations can be utilized to predict future disease burden and establish preventive priorities. Reducing exposure to etiologic factors associated with high-risk behaviors in well designed preventive and health promotion initiatives may contribute to a reduction of existing cancer disparities as well as reducing future disease burden.

Thus, tissue specific global methylation was shown for oral cancer cases with different etiologies. Smoking was correlated to DNA methylation levels when generalized linear model simulations were performed. Future studies should look at global epigenetic alterations associated to the progression from normal to premalignant tissue of oral cancer patients with different etiologies in a case control study.

### Example 3

### The Biomarkers System

The biomarker system consists of a multiplexed panel of global epigenetic biomarkers that can be used by themselves or in combination with other molecular markers of disease enabling a molecular system for early detection, clinical management and recurrence monitoring. The multiplexed panel of epigenetic biomarkers that are enabled in this invention provides a useful molecular tool to fats track the biomarker development trial system set up by the National Cancer Institute Early Detection Research Network (Pepe, 2001).

### Phase 1—Preclinical Exploratory Studies

Phase 1 preclinical exploratory studies were conducted to test the epigenetic biomarker's ability to discriminate between disease and non-disease, comparing tumor tissue with non-tumor tissue as described in example 1.

### Phase 2—Clinical Assay Development For Clinical Disease

Clinical assay development studies are based on a specimen that can be obtained non-invasively (e.g. blood, saliva, tears, urine, cervical smear, ductal lavage fluid, cerebrospinal fluid, serosal fluid, lymph fluid, bile and stool). The clinical assay can distinguish subjects with cancer from those without cancer. Individual specificity and sensitivity of each marker for each cancer site is assessed using receiver operating characteristic (ROC) curves on invasive specimens.

### Phase 3—Retrospective Longitudinal Repository Studies

Tumor banks that have stored DNA or tissue and blood derived products (cells, plasma, etc) and have links to medical records, are utilized to develop a retrospective longitudinal repository study on each cancer site that allows the determination of what environmental, contextual and clinical factors modify the risk of developing an epigenetic alteration associated with cancer for each cancer site.

Phase 4—Prospective Early detection Studies

Appropriate screening study populations are identified and invited to participate in prospective early detection studies, with the assistance of the personnel at the US National Cancer Institute's, Early Research Detection Network and at the Center for Disease Control Office of Public Health Genomics, located in Atlanta.

### Phase 5—Cancer Control Studies

Appropriate screening study populations are identified and invited to participate in prospective screening studies.

### Summary of Biomarker Development

During the Phase 1 and preliminary Phase 2 global methylation work is focused on developing a baseline global methylation index for normal tissue through a series of comparative tests against tissue with histologically confirmed premalignant lesions, with and without dysplasia; metaplastic lesions; and carcinogenic tissue with different degrees of malignancy (example 1).

The cancer site (organ) specific global DNA methylation index can discriminate between normal and different histologic types of transformed tissue along the well characterized continuum of oncogenic progression, from normal to malignant cells. A similar process is being systematically done in Phase 1 and preliminary Phase 2 studies to develop a global histone H4 acetylation index and a global histone H4 methylation index, which can discriminate between normal and different histologic types of transformed tissue along the well characterized continuum of oncogenic progression, from normal to malignant cells. Presently, we are developing and evaluating each epigenetic index separately.

### Example 4

### Epigenetic Biomarkers for Oral and Lung Cancers

Buccal cell isolates serving as viable sources of biomarkers, complementary to traditional sources such as serum or plasma, are utilized for the identification of early cancer and subjects at risk of developing cancer, as a normal cell progresses through the complex process of transformation to a cancerous state. Whole genome epigenetic patterns associated with oral neoplastic lesions are identified. Genome wide epigenetic patterns of DNA methylation and histones H3 and H4 methylation and de-acetylation patterns as early biomarkers of oral carcinogenesis are utilized.

In the present example, the multiplexed biomarker panel is used in the early detection of the following three cancer types: oral squamous cell carcinoma, esophageal squamous cell carcinoma, and lung squamous cell carcinoma. The analytic sensitivity and specificity is estimated within an accuracy of 1% with 95% confidence for each type at each phase of biomarker development trials. Appropriate sample size and power calculations are performed for each participating site and organ of interest using an alpha of 0.01 and a power of 0.95.

A Phase 5 biomarker development trial is performed in this representative longitudinal cohort of 800 individuals designed to test the multiplexed panel developed for oral cancer in the previous four phases.

The biomarkers as described herein provide that global DNA methylation levels differ in normal, premalignant and tumor tissue samples obtained from the oral cavity, esophagus and lung. A DNA hypomethylation gradient is observed across smoking history and tissue type; 2) Global histone H4 acetylation and methylation levels increase the positive predictive value of the global methylation index for those samples with overlapping values between categories, across smoking history and tissue type; 3) Aerodigestive gene expression profiles correlate with the global DNA methylation index and the global histone H4 acetylation and methylation levels across smoking history and tissue type

### Solexa Sequencing Technology

a) Preparation of full diversity libraries of whole genomes. Solexa's oligonucleotide adapters are ligated onto the fragments, yielding a fully-representative genomic library of DNA templates without cloning.

b) Generating a Clonal Single Molecule Array™ Flow Cell. Single molecule clonal amplification involves six steps: Template hybridization, template amplification, linearization, blocking 3′ ends, denaturation and primer hybridization. Flowcell preparation is fully automated on a Solexa Cluster Station creating up to 1000 identical copies of each single molecule achieving densities of up to 10 million clonal clusters per square centimeter.

c) Sequencing-by-Synthesis. The Solexa 1G Genetic Analyzer completely automates Sequencing-by-Synthesis (SBS). The flowcell from step b is loaded into the analyzer. Reagents and buffers in Solexa's SBS kit are placed into the reagent ports and sequencing commences by initiating the instrument software. Solexa's Sequencing-by-Synthesis utilizes four proprietary nucleotides possessing reversible fluorophore and termination properties. Each sequencing cycle occurs in the presence of all four nucleotides leading to higher accuracy than methods where only one nucleotide is present in the reaction mix at a time.

d) Image processing and sequence alignment. Solexa's software suite includes the full range of data collection, processing and analysis modules to streamline collection and analysis of data with minimal user intervention. After sequencing has completed, the data files are mirrored to an off-instrument computer for analysis using a standard pipeline of software that sequentially performs image analysis, generation of base-calls, per-base confidence scores and realignment against a reference database.

MeDIP. Four to eight micrograms of genomic DNA extracted are used produce random fragments ranging in size from 300 to 600 bp. The DNA is denatured for 10 min at 95° C. and immunoprecipitated overnight at 4° C. with 10 μL of monoclonal antibody against 5-methylcytidine (1 mg/mL; Eurogentec). Immunoprecipitated methylated DNA is labeled with Cy5 fluorophere and the input genomic DNA was labeled with Cy3 fluorophere. Labeled DNA from the enriched and the input pools are combined (1-2 μg) and hybridized to the Human CpG Island-Plus-Promoter Array (Roche-Nimblegen), which covers all UCSC-annotated CpG islands and promoter regions for all RefSeq genes.

HELP. One microgram of each DNA sample is digested to completion overnight using HpaII or MspI. The quality of digestion is assessed using gel electrophoresis. One-tenth of the digested sample is added to T4 DNA ligase and the following oligonucleotide pair: JHpaII 12 (6 OD/ml) 7.5, and JHpaII 24 (12 OD/ml) 7.5. The reaction mix is placed in a thermocycler for 5 min at 55° C. then ramped over 1 h to 4° C., at which time 1 unit of T4 DNA ligase is added for overnight ligation at 16° C. The HpaII and MspI representations is cohybridized to a HELP microarray in the Roche-NimbleGen Service Laboratory and scanned to quantify the 532 and 635 nm fluorescence at each oligonucleotide on the microarray.

Transposons insertion site profiling chip (TIP-chip). A genome-wide method for identifying all transposons in any given sample is utilized. This platform is a transposons insertion site profiling chip (TIP-chip), a microarray intended for use as a high-throughput technique for mapping transposons insertions. The TIP-chip method provides genome-wide insertion site preferences and the locations of transposons “hotspots” or “cold spots” associated with environmental exposures in samples from the upper aerodigestive tract.

Global and gene-specific methylation assays. Global DNA methylation is performed using Epigentek's global DNA methylation kit, an ELISA based method to correlate to the HPLC-based on the global DNA methylation assay developed in Manel Esteller's laboratory that we used in our earlier liver cancer work.

Gene-specific methylation assay. Gene-specific methylation analyses is done utilizing bisulfite converted DNA which is amplified and quantified using the quantitative methylation specific PCR (QMSP).

Microarray data acquisition. Six to eight micrograms of total RNA from bronchial epithelial cells are converted into double-stranded cDNA with the SuperScript II reverse transcriptase (Invitrogen) with an oligo-dT primer containing a T7 RNA polymerase promoter (Genset, Boulder, Colo.). The ENZO Bioarray RNA transcript labeling kit (Affymetrix) is used for in vitro transcription of the purified double-stranded cDNA. Each verified cRNA sample is hybridized overnight onto the Affymetrix HG-U133A array, and confocal laser scanning (Agilent) is performed to detect the streptavidin-labeled fluor. A single weighted mean expression level for each gene along with a P (detection) value (which indicates whether the transcript was reliably detected) is derived by using MICROARRAY SUITE 5.0 software (Affymetrix, Santa Clara, Calif.)

Descriptive statistics showing univarible, bi-variable and multivariable characteristics are performed utilizing Stata version 10.0 and R 2.6. Hypothesis testing is preceded by descriptive statistics, including examination of frequency distributions and measures of central tendency and variation, simple correlations among variables, and their variance covariance matrix. Values are log transformed as needed to normalize the distributions. The main hypotheses use an p<0.05 and a 2-tailed test unless otherwise stated.

The relationship between a continuous measure of global DNA methylation (Met), age, current smoking status, former smoking status, and the interaction between former smoking status and months elapsed since quitting smoking (form.tg) are examined with the following sets of equations:
Former smokers Metᵢ=β₀+x_{age}*β_{age}+x_{curr}*β_{curr}+x_{form}*β_{form}+x_{form.tg}*β_{form.tg}+εᵢ
Current smokers Metᵢ=β₀+x_{age}*β_{age}+x_{curr}*β_{curr}*1+εᵢ
Never smokers Metᵢ=β₀+x_{age}*β_{age}+εᵢ
   where εᵢ represents the normally distributed error. Similar models are fit for global histones H4 acetylation and methylation values, gene expression results, genome-wide measures of DNA methylation and histone modifications. Given that the logistic regression and ROC approaches may give contradictory results on the same data, the plot predictiveness curve for each individual biomarker and panels of biomarkers is provided.

A rich amount of data describing the relationship between smoking and DNA hypomethylation in the aerodigestive tract at global, genome-wide and gene specific levels, using an integrative approach that combines epigenomic, and transcriptomic information to understand the underlying biology and develop a multiplexed biomarker for early cancer detection is provided.

The method as described herein represents a paradigmatic shift in the understanding of oncogenesis, from a monoclonal to an epigenetic origin, a break with current thinking in cancer biology. Another conceptual innovation in this project is the use of nucleotide based sequencing technology to study the environmental determinants of global epigenetic regulation in cancer. This project provides global, genome-wide, gene-specific and base-specific epigenetic alterations, triggered as responses to noxious environmental stimuli account for the initial cellular changes in the transition from a normal to a transformed cell, can be used as detection, including early detection, markers for most solid and hematologic tumors.

The method as described herein propose an innovative, fast-track approach to biomarker development by combining advanced basic science tools with novel sampling and cohort selection strategies that ensures a quick transition from the basic science laboratory to the clinic and to populations.

### Example 5

### Epigenetic Biomarkers for Colon Cancer

DNA hypomethylation is a common trait of colorectal cancer. Studies in tumor cell lines and animal models indicate that genome-wide demethylation may cause genetic instability and hence facilitate or accelerate tumor progression. DNA hypomethylation precedes genomic damage in human gastrointestinal cancer, but the nature of this damage has not been clearly established. Here, we show a thorough analysis of DNA methylation and genetic alterations in two series of colorectal carcinomas (Table 2).

TABLE 2
Genomic damage and hypomethylation index in colorectal cancer

| | Series HSP | | | Series HUB | | |
|---|---|---|---|---|---|---|
| | | | Hypomethylation | | No. | Hypomethylation |
| | n | GDF | index | n | alterations | Index |
| All tumors | 83 | 0.172 ± 0.085 | 0.149 ± 0.082 | 50 | 6.8 ± 4.8 | 0.122 ± 0.062 |
| | | (0.015-0.389) | (0.005-0.356) | | (0-16) | (0.017-0.272) |
| p53 status | | | | | | |
| Wild-type | 46 | 0.150 ± 0.082 | 0.142 ± 0.076 | 16 | 3.7 ± 4.1 | 0.102 ± 0.058 |
| Mutated | 33 | 0.202 ± 0.080 | 0.164 ± 0.091 | 32 | 8.4 ± 4.3 | 0.128 ± 0.061 |
| | | P = 0.007 | P = 0.245 | | P = 0.001 | P = 0.162 |

NOTE:
Values are expressed as mean ± SD. Numbers in parentheses indicate range. P values are estimated from two-tailed t test.

The extent of DNA demethylation but not of hypermethylation (both analyzed by amplification of intermethylated sites in near 200 independent sequences arbitrarily selected) correlated with the cumulated genomic damage assessed by two different techniques (arbitrarily primed PCR and comparative genomic hybridization).

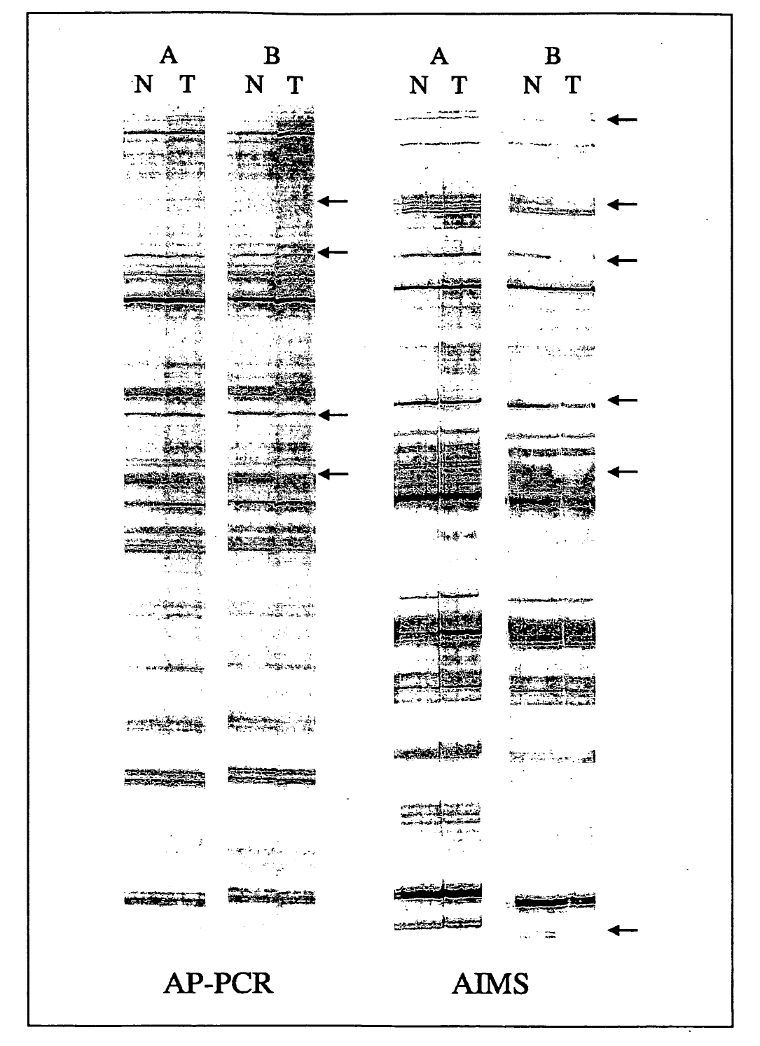

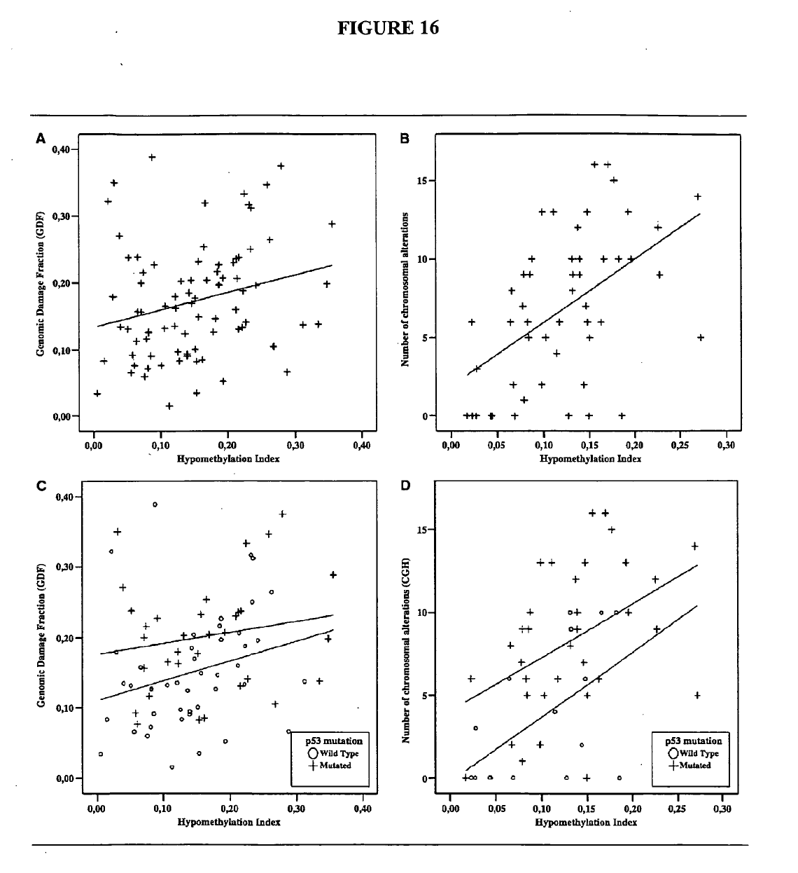

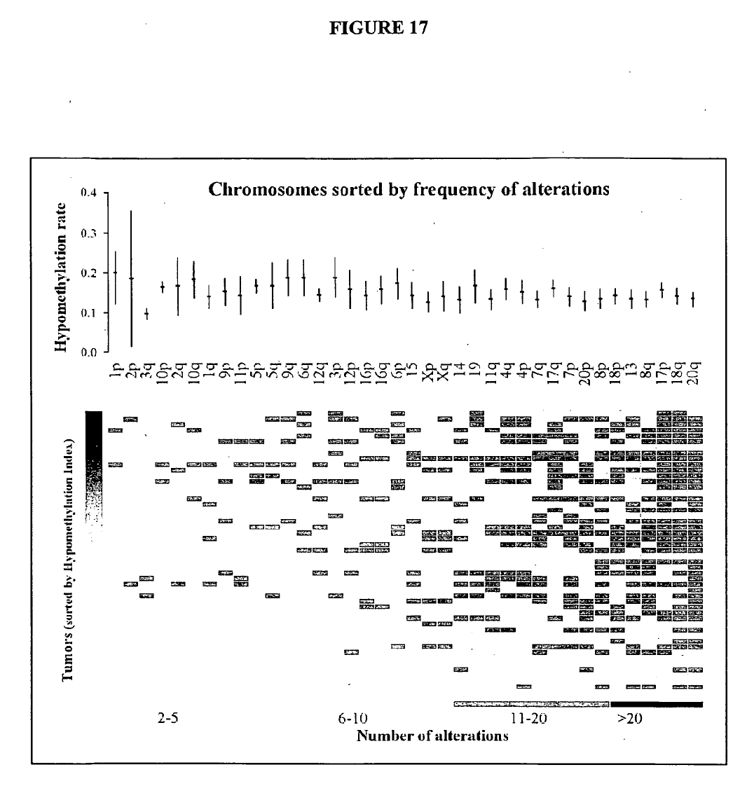

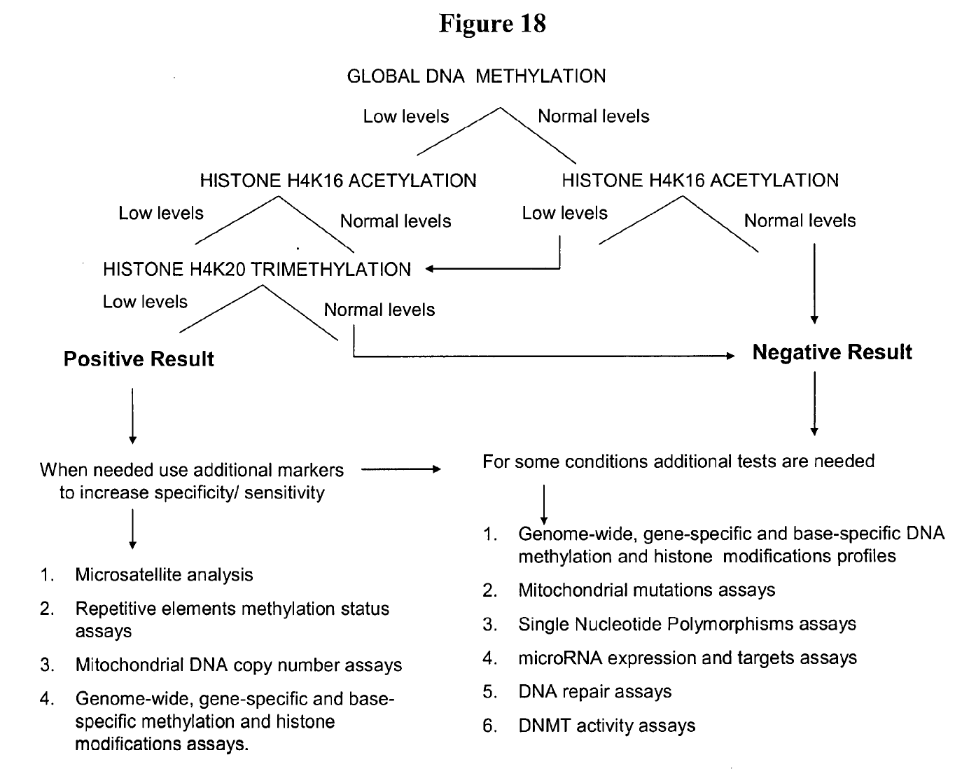

DNA hypomethylation-related instability was mainly of chromosomal nature and could be explained by a genome-wide effect rather than by the concurrence of the most prevalent genetic and epigenetic alterations (FIG. 15). Moreover, the association of p53 mutations with genomic instability was secondary to DNA hypomethylation and the correlation between DNA hypomethylation and genomic instability was observed in tumors with and without mutation in the p53 gene (FIG. 16). Our data support a direct link between genome-wide demethylation and chromosomal instability in human colorectal carcinogenesis and are consistent with the studies in model systems demonstrating a role of DNA demethylation in inducing chromosomal instability (FIG. 17).

### Example 6

### Workflow Flowchart

This example is directed to a workflow flowchart for a multiplexed panel of global DNA methylation and histone modifications assays for early detection, clinical management and disease monitoring (see FIG. 18).

The first test of this multiplexed panel is the global methylation test, followed by a histone H4 Lysine 16 acetylation assay. If a sample has negative results for these two markers then the results of the test are deemed negative. Otherwise, a histone H4 Lysine 20 methylation assay is performed. If the results of the three test are positive (low methylation, low acetylation and low trimethylation levels), then the test is deemed to be positive. If the results of these three tests are a combination of positive and negative results, then an algorithm is used to classify the results, taking into consideration the tissue/cell/exposure specific continuous value for each test, the ratios between the values and the different permutations between positive and negative results for these three tests, clinical and demographic characteristics. The algorithm calculates the probability of a positive result. Additional molecular assays may be needed to increase the sensitivity or specificity of the result: Microsatellite analysis; Repetitive elements methylation status assays; Mitochondrial DNA copy number assays; Genome-wide, gene-specific and base-specific methylation and histone modifications assays.

For some conditions and disease states additional molecular tests are needed. These can vary from genome-wide (Jacinto et al, 2008; Irizarry et al, 2008; Mikkelsen et al, 2007; Bock et al, 2007; Barski et al, 2007; Wheelan et al, 2006), gene-specific (Brock et al, 2008; Schuebel et al, 2007; and base specific DNA methylation and histone modification assays; (Cokus et al, 2008; Kim et al, 2008; Chen et al, 2008; Meyer, 2007; Hafner et al, 2008; Schones et al, 2008) to mitochondrial mutational assays (Dasgupta et al, 2008; Voss et al, 2008; Maitra, 2004), Single Nucleotide Polymorphisms assays (Sanders et al, 2008; Johnson et al, 2007; Macgregor et al, 2008), microRNA expression and target assays, (Hsu et al 2008; Wang and Cheng, 2008; Perera 2007; Castoldi et al, 2007) DNA repair activity assays and DNA Methyltransferases activity (DNMT) assays. These assays can be done in samples that give positive and negative results using multiple technological platforms (Yan et al, 2008; Johnson et al, 2008; Mock et al, 2008; King et al, 2008; Jain, 2007; Brena et al, 2006).

### Example 7

### Global DNA Methylation Values in Saliva from Premalignant Oral Cancer Patients and Controls

Saliva samples were collected from 5 pairs of matched cases and controls between October and December 2006 at the Dental Clinic of the European University of Madrid as part of a collaborative study between the University of Puerto Rico, Columbia University, the Spanish National Cancer Research Center (CNIO) and the European University of Madrid. Dr Luis Alberto Moreno form EUM and Dr Rafael Guerrero-Preston from Columbia University are the Principal Investigators of this project. IRB approval was obtained from the UPR prior to the beginning of sample collection. There were six men and four women in this proof of principle study. Average age was 56. The age range was 49, the maximum being 85 and the minimum 36.

### Sample Collection and DNA Extraction.

Saliva samples (5 ml) were collected in 50 ml conical tubes, frozen immediately and stored at −20 C until transported to the Cancer Epigenetics Laboratory of CNIO. Upon arrival samples were thawed, washed three times with a solution of PBS a cocktail of protease inhibitors (Complete, Roche). The resultant pellet was used for protein and DNA extraction. DNA was extracted utilizing standard a phenol-chloroform extraction protocol and precipitated with ethanol. Quantification was obtained with a Nanodrop.

### Global DNA Methylation Quantification

An ELISA-like test kit was utilized to quantify the global DNA methylation content in these samples (Epigentek, Brooklyn, N.Y.). One hundred nanograms of DNA are immobilized to a strip well specifically coated with DNA affinity substance. The methylated fraction of DNA is recognized by 5-methylcytosine antibody and quantified through an ELISA-like reaction. The amount of methylated DNA is proportional to the OD intensity.

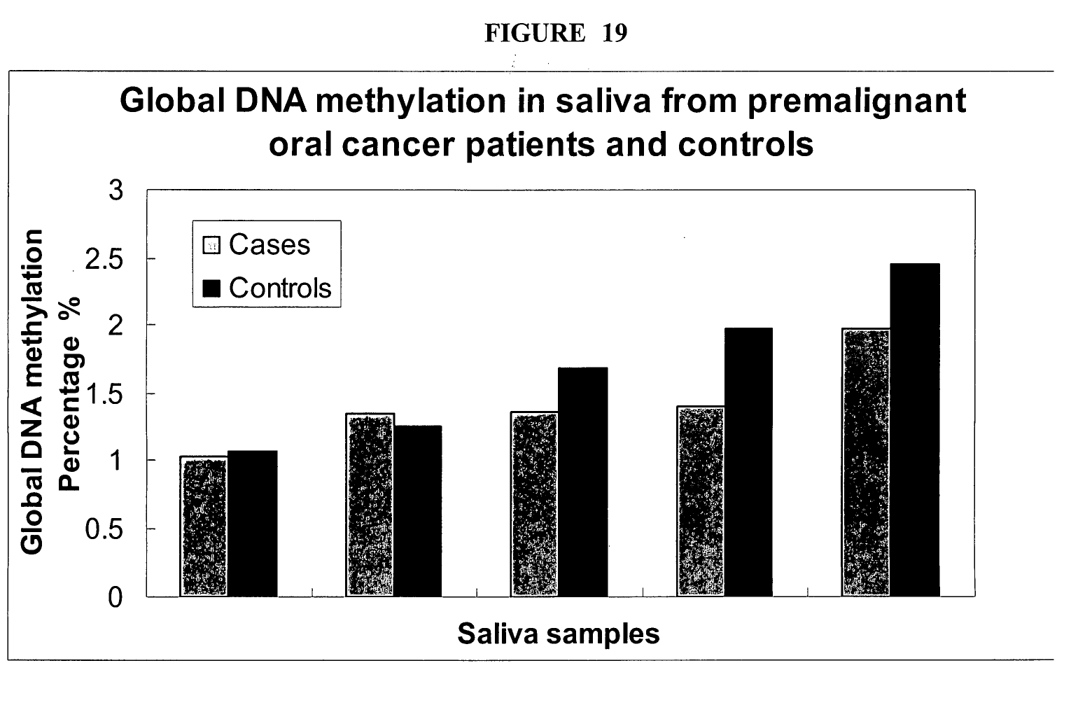

As shown in FIG. 19, three of the five pairs of samples showed a clear difference of DNA methylation between the cases and the controls. As expected, cases showed a lower content of global DNA methylation than the controls. Furthermore, the results from this initial test show that it is possible to utilize saliva as a non-invasive source of global DNA methylation to measure the presence of an oncogenic process in the oral cavity. This is the first time that global DNA methylation has successfully distinguished between premalignant oral cancer cases and controls in DNA extracted from saliva.

### Example 8

### Genomic DNA Methylation as a Biomarker for Bladder Cancer: Case-Control

DNA hypomethylation has been suggested to cause genomic instability and increase cancer risk. We aimed to test the hypothesis that DNA hypomethylation is associated with bladder cancer (Moore, 2008).

We measured cytosine methylation (5-mC) content in genomic DNA from blood cells from patients with bladder cancer enrolled in a large case-control study in Spain between Jan. 1, 1998, and Dec. 31, 2001. Cases were men and women with newly diagnosed and histologically confirmed urothelial carcinoma of the bladder. Controls were selected from patients admitted to the same hospital for diseases or conditions unrelated to smoking or other known risk factors for bladder cancer. Controls were individually matched to cases on age (within 5 years), sex, race, and area of hospital referral.

5-mC content was measured in leucocyte DNA by use of a combination of high-performance capillary electrophoresis, Hpa II digestion, and densitometry. Data on demographics, 34 polymorphisms in nine folate metabolism genes, and nutritional intake of six B vitamins (including folate), alcohol, and smoking were assessed as potential confounders. Relative 5-mC content was expressed as a percentage (% 5-mC) with respect to the total cytosine content (the sum of methylated and non-methylated cytosines). The primary endpoint was median % 5-mC DNA content.

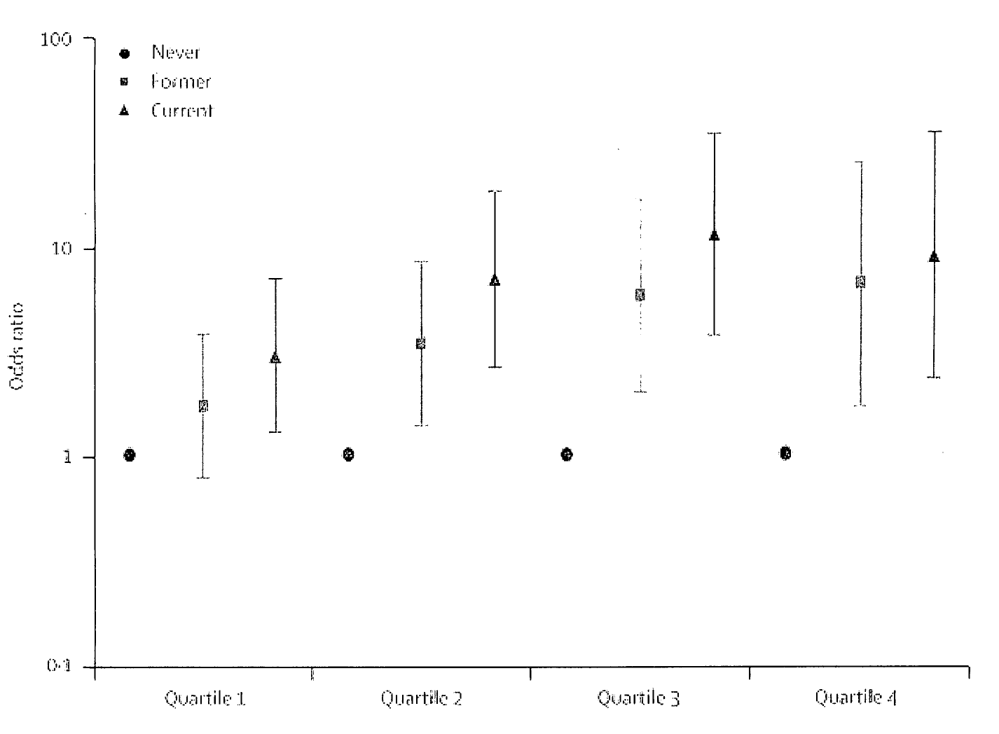

% 5-mC was measured in leucocyte DNA from 775 cases and 397 controls. Median % 5-mC DNA was significantly lower in cases (3.03% [IQR 2.17-3.56]) than in controls (3.19% [2.46-3.68], p=0.0002). All participants were subsequently categorised into quartiles by % 5-mC content in controls. When the highest quartile of % 5-mC content was used as the reference category (Q4), the following adjusted odds ratios (OR) and 95% CI were recorded for decreasing methylation quartiles: OR (Q3) 2.05 (95% CI 1.37-3-06); OR (Q2) 1.62 (1.07-2.44); and OR (Q1) 2.67 (1.77-4.03), p for trend <0.0001. The lowest cancer risk was noted in never smokers in the highest methylation quartile (never smokers in Q4). By comparison with never smokers in the highest quartile, current smokers in the lowest methylation quartile had the highest risk of bladder cancer (Q1: OR 25.51 [9.61-67-76], p for interaction 0.06) (FIG. 20).

### Example 9

### Epigenetic Biomarkers for Other Diseases

Global DNA methylation also occupies a place at the crossroads of many pathways other diseases such as in immunology, where it provides us with a clearer understanding of the molecular network of the immune system. From the classical genetic standpoint, two immunodeficiency syndromes, the ICF (immunodeficiency-centromeric regions instability-facial anomalies) and ATRX (X-linked form of syndromal retardation associated with alpha thalassemia) syndromes, are caused by germline mutations in two epigenetic genes: the DNA methyltransferase DNMT3b and the ATRX genes. Autoimmunity and DNA methylation can also go hand in hand. Classical autoimmune diseases, such as systemic lupus erythematosus or rheumatoid arthritis, are characterized by massive genomic hypomethylation. This phenomenon is highly reminiscent of the global demethylation observed in the DNA of cancer cells compared with their normal-tissue counterparts. Several other examples are also worth mentioning, such as the proposed epigenetic control of the histo-blood group ABO genes and the silencing of human leukocyte antigen (HLA) class I antigens.

In addition, aberrant global, genome-wide, gene-specific and base-specific epigenomic, DNA methylation and histones modification patterns go beyond the fields of oncology and immunology to touch a wide range of fields of biomedical and scientific knowledge. In neurology and autism research, for example, it was surprising to discover that germline mutations in the methyl-binding protein MeCP2 (a key element in the silencing of gene expression mediated by DNA methylation) causes the common neurodevelopmental disease known as Rett syndrome. DNA methylation and chromatin conformation alterations underlie other, more prevalent neurological pathologies, such as schizophrenia or Alzheimer's disease. Beyond that, DNA methylation changes are also known to be involved in cardiovascular disease, the biggest killer in western countries. For example, aberrant CpG island hypermethylation has been described in atherosclerotic lesions. Germline variants and mutations in genes involved in the metabolism of the methyl-group (such as MTHFR) cause changes in DNA methylation, and changes in the levels of methyl-acceptors and methyl-donors are responsible for the pathogenesis of diseases related to homocysteinemia and spina bifida. Imprinting disorders, which represent another huge area of research, are the perfect example of methylation-dependent epigenetic human diseases. A perfectly confined DNA methylation change causes Beckwith-Wiedemann syndrome, Prader-Willi/Angelman syndromes, Russell-Silver syndrome and Albright hereditary osteodystrophy. This highlights the absolute necessity to maintain the correct DNA methylome in order to achieve harmonized development.

The emergence of a new technology for studying DNA methylation based on bisulfite modification coupled with PCR has been decisive in the expansion of the field. The popularization of the bisulfite treatment of DNA (which changes unmethylated ‘C’ to ‘T’ but maintains the methylated ‘C’ as a ‘C’), associated with amplification by specific polymerase chain reaction primers (methylation-specific polymerase chain reaction), Taqman, restriction analysis and genomic sequencing has made it possible for every laboratory and hospital in the world to have a fair opportunity to study DNA methylation, even using pathological material from old archives. We may call this change the ‘universalization of DNA methylation’. The techniques described, which are ideal for studying biological fluids and the detailed DNA methylation patterns of particular tumor suppressor genes, can also be coupled with global genomic approaches for establishing molecular signatures of tumors based on DNA methylation markers, such as CpG island microarrays, (Mill et al, 2008; Shen et al, 2007) Restriction Landmark Genomic Scanning and Amplification of Intermethylated Sites (Matsuyama, 2008; Wnag et al, 2008; Shivapurkar et al, 2008).

These canonical technologies developed in cancer epigenetics are now been used to understand other diseases and physiologic processes.

### REFERENCES

1. Boix-Chornet M, Fraga M F, Villar-Garea A, Caballero R, Espada J, Nuñez A, Casado J, Largo C, Casal J I, Cigudosa J C, Franco L, Esteller M, Ballestar E. Release of hypoacetylated and trimethylated histone H4 is an epigenetic marker of early apoptosis. J Biol. Chem. 2006 May 12; 281(19):13540-7. Epub 2006 Mar. 10.
2. Brena R M, Auer H, Kornacker K, Plass C. Quantification of DNA methylation in electrofluidics chips (Bio-COBRA). Nat. Protoc. 2006; 1(1):52-8.
3. Carvalho A L, Jeronimo C, Kim M M, Henrique R, Zhang Z, Hoque M O, Chang S, Brait M, Nayak C S, Jiang W W, Claybourne Q, Tokumaru Y, Lee J, Goldenberg D, Garrett-Mayer E, Goodman S, Moon C S, Koch W, Westra W H, Sidransky D, Califano J A. Evaluation of promoter hypermethylation detection in body fluids as a screening/diagnosis tool for head and neck squamous cell carcinoma. Clin Cancer Res. 2008 Jan. 1; 14(1):97-107.
4. Ellinger J, Bastian P J, Jurgan T, Biermann K, Kahl P, Heukamp L C, Wernert N, Müller S C, von Ruecker A. CpG island hypermethylation at multiple gene sites in diagnosis and prognosis of prostate cancer. Urology. 2008 January; 71(1):161-7.
5. Feng Q, Hawes S E, Stern J E, Dem A, Sow P S, Dembele B, Toure P, Soya P, Laird P W, Kiviat N B. Promoter hypermethylation of tumor suppressor genes in urine from patients with cervical neoplasia. Cancer Epidemiol Biomarkers Prey. 2007 June; 16(6):1178-84.
6. Fraga M F, Ballestar E, Paz M F, Ropero S, Setien F, Ballestar M L, Heine-Suñer D, Cigudosa J C, Urioste M, Benitez J, Boix-Chornet M, Sanchez-Aguilera A, Ling C, Carlsson E, Poulsen P, Vaag A, Stephan Z, Spector T D, Wu Y Z, Plass C, Esteller M. Epigenetic differences arise during the lifetime of monozygotic twins. Proc Natl Acad Sci USA. 2005 Jul. 26; 102(30):10604-9. Epub 2005 Jul. 11.
7. Fraga M F, Ballestar E, Villar-Garea A, Boix-Chornet M, Espada J, Schotta G, Bonaldi T, Haydon C, Ropero S, Petrie K, Iyer N G, Pérez-Rosado A, Calvo E, Lopez J A, Cano A, Calasanz M J, Colomer D, Piris M A, Ahn N, Imhof A, Caldas C, Jenuwein T, Esteller M. Loss of acetylation at Lys16 and trimethylation at Lys20 of histone H4 is a common hallmark of human cancer. Nat. Genet. 2005a April; 37(4):391-400.
8. Fraga M F, Herranz M, Espada J, Ballestar E, Paz M F, Ropero S, Erkek E, Bozdogan O, Peinado H, Niveleau A, Mao J H, Balmain A, Cano A, Esteller M. A mouse skin multistage carcinogenesis model reflects the aberrant DNA methylation patterns of human tumors. Cancer Res. 2004b Aug. 15; 64(16):5527-34.
9. Frigola J, Solé X, Paz M F, Moreno V, Esteller M, Capella G, Peinado M A.
10. Differential DNA hypermethylation and hypomethylation signatures in colorectal cancer. Hum Mol. Genet. 2005 Jan. 15; 14(2):319-26. Epub 2004 Dec. 1.
11. Guerrero-Preston R, Santella R M, Blanco A, Desai M, Berdasco M, Fraga M. Global DNA hypomethylation in liver cancer cases and controls: a phase I preclinical biomarker development study. Epigenetics. 2007 October-December; 2(4):223-6.
12. Honrado E, Osorio A, Milne R L, Paz M F, Melchor L, Cascón A, Urioste M, Cazorla A, Díez O, Lerma E, Esteller M, Palacios J, Benitez J. Immunohistochemical classification of non-BRCA1/2 tumors identifies different groups that demonstrate the heterogeneity of BRCAX families. Mod Pathol. 2007 December; 20(12):1298-306. Epub 2007 Sep. 21.
13. Hsiung D T, Marsit C J, Houseman E A, Eddy K, Furniss C S, McClean M D, Kelsey K T. Global DNA methylation level in whole blood as a biomarker in head and neck squamous cell carcinoma. Cancer Epidemiol Biomarkers Prey. 2007 January; 16(1):108-14.
14. Lockwood W W, Chari R, Coe B P, Girard L, Macaulay C, Lam S, Gazdar A F, Minna J D, Lam W L. DNA amplification is a ubiquitous mechanism of oncogene activation in lung and other cancers. Oncogene. 2008 Apr. 7 [Epub ahead of print]
15. Moore L E, Pfeiffer R M, Poscablo C, Real F X, Kogevinas M, Silverman D, Garcia-Closas R, Chanock S, Tardon A, Serra C, Carrato A, Dosemeci M, Garcia-Closas M, Esteller M, Fraga M, Rothman N, Malats N. Genomic DNA hypomethylation as a biomarker for bladder cancer susceptibility in the Spanish Bladder Cancer Study: a case-control study. Lancet Oncol. 2008 April; 9(4):359-66. Epub 2008 Mar. 12.
16. Paz M F, Fraga M F, Avila S, Guo M, Pollan M, Herman J G, Esteller M A systematic profile of DNA methylation in human cancer cell lines. Cancer Res. 2003 Mar. 1; 63(5):1114-21.
17. Pepe M S, Etzioni R, Feng Z, Potter J D, Thompson M L, Thomquist M, Winget M, Yasui Y. Phases of biomarker development for early detection of cancer. J Natl Cancer Inst. 2001 Jul. 18; 93(14):1054-61.
18. Ballestar E, Esteller M. Chapter 9 epigenetic gene regulation in cancer. Adv Genet. 2008; 61:247-67.
19. Dolinoy D C, Jirtle R L. Environmental epigenomics in human health and disease. Environ Mol. Mutagen. 2008 January; 49(1):4-8.
20. Dolinoy D C, Weidman J R, Jirtle R L. Epigenetic gene regulation: linking early developmental environment to adult disease. Reprod Toxicol. 2007 April-May; 23(3):297-307. Epub 2006 Sep. 9.
21. Ehrlich M. Cancer-linked DNA hypomethylation and its relationship to hypermethylation. Curr Top Microbiol Immunol. 2006; 310:251-74.
22. Esteller M. Epigenetics in cancer. N Engl J. Med. 2008 Mar. 13; 358(11):1148-59.
23. Esteller M. Epigenetic gene silencing in cancer: the DNA hypermethylome. Hum Mol. Genet. 2007(a) Apr. 15; 16 Spec No 1:R50-9.
24. Esteller M. Cancer epigenomics: DNA methylomes and histone-modification maps. Nat Rev Genet. 2007(b) April; 8(4):286-98.
25. Esteller M. The necessity of a human epigenome project. Carcinogenesis. 2006(a) June; 27(6):1121-5.
26. Esteller M. Epigenetics provides a new generation of oncogenes and tumour-suppressor genes. Br J. Cancer. 2006(b) Jan. 30; 94(2):179-83.
27. Lujambio A, Ropero S, Ballestar E, Fraga M F, Cerrato C, Setien F, Casado S,
28. Suarez-Gauthier A, Sanchez-Cespedes M, Git A, Spiteri I, Das P P, Caldas C, Miska
29. E, Esteller M. Genetic unmasking of an epigenetically silenced microRNA in human cancer cells. Cancer Res. 2007(a) Feb. 15; 67(4):1424-9.
30. Lujambio A, Esteller M. CpG island hypermethylation of tumor suppressor microRNAs in human cancer. Cell Cycle. 2007(b) Jun. 15; 6(12):1455-9.
31. Meaney M J, Szyf M. Environmental programming of stress responses through DNA methylation: life at the interface between a dynamic environment and a fixed genome. Dialogues Clin Neurosci. 2005; 7(2):103-23.
32. Meaney M J, Szyf M.
33. Fraga M F, Esteller M Epigenetics and aging: the targets and the marks. Trends Genet. 2007(a) August; 23(8):413-8.
34. Fraga M F, Agrelo R, Esteller M. Cross-talk between aging and cancer: the epigenetic language. Ann N Y Acad. Sci. 2007(b) April; 1100:60-74.
35. Herranz M, Esteller M. DNA methylation and histone modifications in patients with cancer: potential prognostic and therapeutic targets. Methods Mol. Biol. 2007; 361:25-62.
36. Hoffmann M J, Schulz W A. Causes and consequences of DNA hypomethylation in human cancer. Biochem Cell Biol. 2005 June; 83(3):296-321.
37. Sinclair K D, Allegrucci C, Singh R, Gardner D S, Sebastian S, Bispham J, Thurston
38. A, Huntley J F, Rees W D, Maloney C A, Lea R G, Craigon J, McEvoy T G, Young L E. DNA methylation, insulin resistance, and blood pressure in offspring determined
39. by maternal periconceptional B vitamin and methionine status. Proc Natl Acad Sci USA. 2007 Dec. 4; 104(49):19351-6.
40. Szyf M, McGowan P, Meaney M J. The social environment and the epigenome. Environ Mol. Mutagen. 2008 January; 49(1):46-60.
41. Szyf M. The dynamic epigenome and its implications in toxicology. Toxicol Sci. 2007 November; 100(1):7-23. Epub 2007 Aug. 3.
42. Wilson A S, Power B E, Molloy P L. DNA hypomethylation and human diseases. Biochim Biophys Acta. 2007 January; 1775(1):138-62.
Eberharter, A. and P. B. Becker, Histone acetylation: a switch between repressive and permissive chromatin. Second in review series on chromatin dynamics. EMBO Rep, 2002. 3(3): p. 224-9.
Esteller, M., Epigenetics provides a new generation of oncogenes and tumour-suppressor genes. Br J Cancer, 2006. 94(2): p. 179-83.
Fraga, M. F., et al., A mouse skin multistage carcinogenesis model reflects the aberrant DNA methylation patterns of human tumors. Cancer Res, 2004. 64(16): p. 5527-34.
Fraga, M. F. and M. Esteller, Towards the human cancer epigenome: a first draft of histone modifications. Cell Cycle, 2005c. 4(10): p. 1377-81.
69. Stahl, B. D. and C. D. Allis, The language of covalent histone modifications. Nature, 2000. 403(6765): p. 41-5.
70. Cheung, R. C., et al., Chronic Hepatitis C in Latinos: Natural History, Treatment Eligibility, Acceptance, and Outcomes. The American Journal of Gastroenterology, 2005. 100: p. 2186-2193.
71. Eberharter, A. and P. B. Becker, Histone acetylation: a switch between repressive and permissive chromatin. Second in review series on chromatin dynamics. EMBO Rep, 2002. 3(3): p. 224-9.
72. Studnicki, J., et al., Hispanic health status in Orange County, Florida. J Public Health Manag Pract, 2005. 11(4): p. 326-32.
73. Tycko, B., Epigenetic gene silencing in cancer. J Clin Invest, 2000. 105(4): p. 401-7.
79. Zhang, Y. J., et al., Predicting hepatocellular carcinoma by detection of aberrant promoter methylation in serum DNA. Clin Cancer Res, 2007. 13(8): p. 2378-84.
80. Seligson, D. B., et al., Global histone modification patterns predict risk of prostate cancer recurrence. Nature, 2005. 435(7046): p. 1262-6.
81. Kaneda, A., et al., Frequent hypomethylation in multiple promoter CpG islands is associated with global hypomethylation, but not with frequent promoter hypermethylation. Cancer Sci, 2004. 95(1): p. 58-64.
82. Baylin, S. B., et al., Alterations in DNA methylation: a fundamental aspect of neoplasia. Adv Cancer Res, 1998. 72: p. 141-96.
83. Feinberg, A. P. and B. Vogelstein, Hypomethylation distinguishes genes of some human cancers from their normal counterparts. Nature, 1983. 301(5895): p. 89-92.
84. Jones, P. A. and S. B. Baylin, The fundamental role of epigenetic events in cancer. Nat Rev Genet, 2002. 3(6): p. 415-28.
85. Eden, A., et al., Chromosomal instability and tumors promoted by DNA hypomethylation. Science, 2003. 300(5618): p. 455.
86. Gaudet, F., et al., Induction of tumors in mice by genomic hypomethylation. Science, 2003. 300(5618): p. 489-92.
87. Matsuzaki, K., et al., The relationship between global methylation level, loss of heterozygosity, and microsatellite instability in sporadic colorectal cancer. Clin Cancer Res, 2005. 11(24 Pt 1): p. 8564-9.
88. Jaffe, L. F., Epigenetic theories of cancer initiation. Adv Cancer Res, 2003. 90: p. 209-30.
89. Goll, M. G. and T. H. Bestor, Eukaryotic cytosine methyltransferases. Annu Rev Biochem, 2005. 74: p. 481-514.
90. Pogribny, I. P., et al., Irreversible global DNA hypomethylation as a key step in hepatocarcinogenesis induced by dietary methyl deficiency. Mutat Res, 2006. 593(1-2): p. 80-7.
91. Chalitchagorn, K., et al., Distinctive pattern of LINE-1 methylation level in normal tissues and the association with carcinogenesis. Oncogene, 2004. 23(54): p. 8841-6.
92. Shen, L., et al., Correlation between DNA methylation and pathological changes in human hepatocellular carcinoma. Hepatogastroenterology, 1998. 45(23): p. 1753-9.
93. Giannelli, G. and S. Antonaci, New frontiers in biomarkers for hepatocellular carcinoma. Dig Liver Dis, 2006. 38(11): p. 854-9.
67. Verma, M. and S. Srivastava, Epigenetics in cancer: implications for early detection and prevention. Lancet Oncol, 2002. 3(12): p. 755-63.
19. Fraga, M. F., et al., Loss of acetylation at Lys16 and trimethylation at Lys20 of histone H4 is a common hallmark of human cancer. Nat Genet, 2005. 37(4): p. 391-400.
94. Dodge, J. E., et al., Inactivation of Dnmt3b in mouse embryonic fibroblasts results in DNA hypomethylation, chromosomal instability, and spontaneous immortalization. J Biol Chem, 2005. 280(18): p. 17986-91.
95. James, S. R., P. A. Link, and A. R. Karpf, Epigenetic regulation of X-linked cancer/germline antigen genes by DNMT1 and DNMT3b. Oncogene, 2006. 25(52): p. 6975-85.
96. Lujambio, A., et al., Genetic unmasking of an epigenetically silenced microRNA in human cancer cells. Cancer Res, 2007. 67(4): p. 1424-9.
97. Costa, F. F., Non-coding RNAs: new players in eukaryotic biology. Gene, 2005. 357(2): p. 83-94.
98. Koturbash, I., I. Pogribny, and O. Kovalchuk, Stable loss of global DNA methylation in the radiation-target tissue—a possible mechanism contributing to radiation carcinogenesis? Biochem Biophys Res Commun, 2005. 337(2): p. 526-33.
99. Pogribny, I., et al., Fractionated low-dose radiation exposure leads to accumulation of DNA damage and profound alterations in DNA and histone methylation in the murine thymus. Mol Cancer Res, 2005. 3(10): p. 553-61.
9. Yu, J., et al., Methylation profiling of twenty promoter-CpG islands of genes which may contribute to hepatocellular carcinogenesis. BMC Cancer, 2002. 2: p. 29.
10. Yu, J., et al., Methylation profiling of twenty four genes and the concordant methylation behaviours of nineteen genes that may contribute to hepatocellular carcinogenesis. Cell Res, 2003. 13(5): p. 319-33.
11. Herath, N. I., B. A. Leggett, and G. A. MacDonald, Review of genetic and epigenetic alterations in hepatocarcinogenesis. J Gastroenterol Hepatol, 2006. 21(1 Pt 1): p. 15-21.
79. Tada, M., et al., Hypermethylation of NAD(P)H: quinone oxidoreductase 1 (NQO1) gene in human hepatocellular carcinoma. J Hepatol, 2005. 42(4): p. 511-9.
100. Lee, S., et al., Aberrant CpG island hypermethylation along multistep hepatocarcinogenesis. Am J Pathol, 2003. 163(4): p. 1371-8.
101. Fang, J. Y., et al., Effects of DNA methylation on expression of tumor suppressor genes and proto-oncogene in human colon cancer cell lines. World J Gastroenterol, 2003. 9(9): p. 1976-80.
102. Calvisi, D. F., et al., Mechanistic and prognostic significance of aberrant methylation in the molecular pathogenesis of human hepatocellular carcinoma. J Clin Invest, 2007. 117(9): p. 2713-22.
103. Schlaeger, C., et al., Etiology-dependent molecular mechanisms in human hepatocarcinogenesis. Hepatology, 2008. 47(2): p. 511-20.
104. Tangkijvanich, P., et al., Serum LINE-1 hypomethylation as a potential prognostic marker for hepatocellular carcinoma. Clin Chim Acta, 2007. 379(1-2): p. 127-33.
105. Winget, M. D., et al., Development of common data elements: the experience of and recommendations from the early detection research network. Int J Med Inform, 2003. 70(1): p. 41-8.
106. Nomoto, S., et al., Hypermethylation of multiple genes as clonal markers in multicentric hepatocellular carcinoma. Br J Cancer, 2007. 97(9): p. 1260-5.
107. Verma, M. and S. Srivastava, New cancer biomarkers deriving from NCI early detection research. Recent Results Cancer Res, 2003. 163: p. 72-84; discussion 264-6.
Lin J C, Jeong S, Lang G, Takai D, Fatemi M, Tsai Y C, Egger G, Gal-Yam E N, Jones Role of nucleosomal occupancy in the epigenetic silencing of the MLH1 CpG island. Cancer Cell. 2007 November; 12(5):432-44.
Mund C, Brueckner B, Lyko F. Reactivation of epigenetically silenced genes by DNA methyltransferase inhibitors: basic concepts and clinical applications. Epigenetics. 2006 January-March; 1(1):7-13.
Shen L, Kondo Y, Ahmed S, Boumber Y, Konishi K, Guo Y, Chen X, Vilaythong J N,
Issa J P. Drug sensitivity prediction by CpG island methylation profile in the NCI-60 cancer cell line panel. Cancer Res. 2007 Dec. 1; 67(23):11335-43.
Wong N I, Lo Y M, Johnson P J. Epigenetic tumor markers in plasma and serum: biology and applications to molecular diagnosis and disease monitoring. Ann N Y Acad. Sci. 2001 September; 945:36-50.
Yang A S, Estécio M R, Doshi K, Kondo Y, Tajara E H, Issa J P. A simple method for estimating global DNA methylation using bisulfite PCR of repetitive DNA elements. Nucleic Acids Res. 2004 Feb. 18; 32(3):e38.
Lecomte T, Berger A, Zinzindohoué F, Micard S, Landi B, Blons H, Beaune P, Cugnenc P H, Laurent-Puig P. Detection of free-circulating tumor-associated DNA in plasma of colorectal cancer patients and its association with prognosis. Int J. Cancer. 2002 Aug. 10; 100(5):542-8.
Miller C A, Campbell S L, Sweatt M. DNA methylation and histone acetylation work in concert to regulate memory formation and synaptic plasticity. Neurobiol Learn Mem. 2007 Sep. 17 [Epub ahead of print]
Miller C A, Sweatt J D. Covalent modification of DNA regulates memory formation. Neuron. 2007 Mar. 15; 53(6):857-69.
Weaver I C. Epigenetic programming by maternal behavior and pharmacological intervention. Nature versus nurture: let's call the whole thing off. Epigenetics. 2007 January-March; 2(1):22-8. Epub 2007 Jan. 15.
Szyf M, Weaver I, Meaney M. Maternal care, the epigenome and phenotypic differences in behavior. Reprod Toxicol. 2007b July; 24(1):9-19. Epub 2007 May 10.
Jacinto F V, Ballestar E, Esteller M. Methyl-DNA immunoprecipitation (MeDIP): hunting down the DNA methylome. Biotechniques. 2008 January; 44(1):35, 37, 39 passim.

### Comment in:

Nature. 2007 Aug. 2; 448(7153):548-9.
Mikkelsen T S, Ku M, Jaffe D B, Issac B, Lieberman E, Giannoukos G, Alvarez P, Brockman W, Kim T K, Koche R P, Lee W, Mendenhall E, O′Donovan A, Presser A, Russ C, Xie X, Meissner A, Wernig M, Jaenisch R, Nusbaum C, Lander E S, Bernstein B E. Genome-wide maps of chromatin state in pluripotent and lineage-committed cells. Nature. 2007 Aug. 2; 448(7153):553-60. Epub 2007 Jul. 1.
Weaver I C, Meaney M J, Szyf M. Maternal care effects on the hippocampal transcriptome and anxiety-mediated behaviors in the offspring that are reversible in adulthood. Proc Natl Acad Sci USA. 2006 Feb. 28; 103(9):3480-5. Epub 2006 Feb. 16.
Bock C, Walter J, Paulsen M, Lengauer T. CpG island mapping by epigenome prediction. PLoS Comput Biol. 2007 June; 3(6):e110. Epub 2007 May 2.

### Comment in:

Cell. 2007 Nov. 30; 131(5):831-2; author reply 832-3.
Barski A, Cuddapah S, Cui K, Roh T Y, Schones D E, Wang Z, Wei G, Chepelev I, Zhao K. High-resolution profiling of histone methylations in the human genome. Cell. 2007 May 18; 129(4):823-37.
Cokus S J, Feng S, Zhang X, Chen Z, Merriman B, Haudenschild C D, Pradhan S, Nelson S F, Pellegrini M, Jacobsen S E. Shotgun bisulphite sequencing of the Arabidopsis genome reveals DNA methylation patterning. Nature. 2008 Mar. 13; 452(7184):215-9. Epub 2008 Feb. 17.
Kim S, Li M, Paik H, Nephew K, Shi H, Kramer R, Xu D, Huang T H. Predicting DNA methylation susceptibility using CpG flanking sequences. Pac Symp Biocomput. 2008; 315-26.
Chen W, Kalscheu V, Tzschach A, Menzel C, Ullmann R, Schulz M, Erdogan F, Li N, Kijas Z, Arkesteijn G, Pajares I L, Goetz-Sothmann M, Heinrich U, Rost I, Dufke A, Grasshoff U, Glaeser B G, Vingron M, Ropers H H. Mapping translocation breakpoints by next-generation sequencing. Genome Res. 2008 Mar. 7 [Epub ahead of print]
Meyer M, Briggs A W, Maricic T, Höber B, Höffner B, Krause J, Weihmann A, Pääbo S, Hofreiter M. From micrograms to picograms: quantitative PCR reduces the material demands of high-throughput sequencing. Nucleic Acids Res. 2008 January; 36(1):e5. Epub 2007 Dec. 15.
Hafner M, Landgraf P, Ludwig J, Rice A, Ojo T, Lin C, Holoch D, Lim C, Tuschl T. Identification of microRNAs and other small regulatory RNAs using cDNA library sequencing. Methods. 2008 January; 44(1):3-12.
Schones D E, Cui K, Cuddapah S, Roh T Y, Barski A, Wang Z, Wei G, Zhao K. Dynamic regulation of nucleosome positioning in the human genome. Cell. 2008 Mar. 7; 132(5):887-98.
Dasgupta S, Hoque M O, Upadhyay S, Sidransky D. Mitochondrial cytochrome B gene mutation promotes tumor growth in bladder cancer. Cancer Res. 2008 Feb. 1; 68(3):700-6.
Maitra A, Cohen Y, Gillespie S E, Mambo E, Fukushima N, Hoque M O, Shah N, Goggins M, Califano J, Sidransky D, Chakravarti A. The Human MitoChip: a high-throughput sequencing microarray for mitochondrial mutation detection. Genome Res. 2004 May; 14(5):812-9.
Sanders M A, Verhaak R G, Geertsma-Kleinekoort W M, Abbas S, Horsman S, van der Spek P J, Löwenberg B, Valk P J. SNPExpress: integrated visualization of genome-wide genotypes, copy numbers and gene expression levels. BMC Genomics. 2008 Jan. 25; 9:41.
Johnson N, Speirs V, Curtin N J, Hall A G. A comparative study of genome-wide SNP, CGH microarray and protein expression analysis to explore genotypic and phenotypic mechanisms of acquired antiestrogen resistance in breast cancer. Breast Cancer Res Treat. 2007 Sep. 28 [Epub ahead of print]
Irizarry R A, Ladd-Acosta C, Carvalho B, Wu H, Brandenburg S A, Wen B, Feinberg A P. Comprehensive high-throughput arrays for relative methylation (CHARM). Genome Res. 2008 Mar. 3 [Epub ahead of print]
Macgregor S, Zhao Z Z, Henders A, Nicholas M G, Montgomery G W, Visscher P M. Highly cost-efficient genome-wide association studies using DNA pools and denseSNP arrays. ucleic Acids Res. 2008 April; 36(6):e35. Epub 2008 Feb. 14.
Hsu S D, Chu C H, Tsou A P, Chen S J, Chen H C, Hsu P W, Wong Y H, Chen Y H, Chen G H,
Huang H D. miRNAMap 2.0: genomic maps of microRNAs in metazoan genomes. Nucleic Acids Res. 2008 January; 36(Database issue).
Wang J W, Cheng J Q. A simple method for profiling miRNA expression. Methods Mol. Biol. 2008; 414:183-90.
Perera R J. A microarray-based method to profile global microRNA expression in human and mouse. Methods Mol. Biol. 2007; 382:137-48.
Castoldi M, Benes V, Hentze M W, Muckenthaler M U. miChip: a microarray platform for expression profiling of microRNAs based on locked nucleic acid (LNA) oligonucleotide capture probes. Methods. 2007 October; 43(2):146-52.
Brock M V, Hooker C M, Ota-Machida E, Han Y, Guo M, Ames S, Glöckner S, Piantadosi S, Gabrielson E, Pridham G, Pelosky K, Belinsky S A, Yang S C, Baylin S B, Herman J G. DNA methylation markers and early recurrence in stage I lung cancer. N Engl J. Med. 2008 Mar. 13; 358(11):1118-28.
Schuebel K E, Chen W, Cope L, Glockner S C, Suzuki H, Yi J M, Chan T A, Van Neste L, Van Criekinge W, van den Bosch S, van Engeland M, Ting A H, Jair K, Yu W, Toyota M, Imai K, Ahuja N, Herman J G, Baylin S B. Comparing the DNA hypermethylome with gene mutations in human colorectal cancer. PLoS Genet. 2007 September; 3(9):1709-23. Epub 2007 Jul. 31.
Voss J G, Raju R, Logun C, Danner R L, Munson P J, Rangel Z, Dalakas M C. A focused microarray to study human mitochondrial and nuclear gene expression. Biol Res Nurs. 2008 April; 9(4):272-9.
Wheelan S J, Scheifele L Z, Martinez-Murillo F, Irizarry R A, Boeke J D. Transposon insertion site profiling chip (TIP-chip). Proc Natl Acad Sci USA. 2006 Nov. 21; 103(47):17632-7. Epub 2006 Nov. 13.
Yan H, Zhang B, Wu H. Chemical cytometry on microfluidic chips. Electrophoresis. 2008 Apr. 2 [Epub ahead of print]
Brena R M, Auer H, Kornacker K, Plass C. Quantification of DNA methylation in electrofluidics chips (Bio-COBRA). Nat. Protoc. 2006; 1(1):52-8.
Johnson C J, Zhukovsky N, Cass A E, Nagy J M. Proteomics, nanotechnology and molecular diagnostics. Proteomics. 2008 February; 8(4):715-30.
Jain K K. Applications of nanobiotechnology in clinical diagnostics. Clin Chem. 2007 November; 53(11):2002-9. Epub 2007 Sep. 21.
Mock T, Samanta M P, Iverson V, Berthiaume C, Robison M, Holtermann K, Durkin C, Bondurant S S, Richmond K, Rodesch M, Kallas T, Huttlin E L, Cerrina F, Sussman M R, Armbrust E V. Whole-genome expression profiling of the marine diatom Thalassiosira pseudonana identifies genes involved in silicon bioprocesses. Proc Natl Acad Sci USA. 2008 Feb. 5; 105(5):1579-84. Epub 2008 Jan. 22.
King K R, Wang S, Jayaraman A, Yarmush M L, Toner M. Microfluidic flow-encoded switching for parallel control of dynamic cellularmicroenvironments. Lab Chip. 2008 January; 8(1):107-16.
Mill J, Tang T, Kaminsky Z, Khare T, Yazdanpanah S, Bouchard L, Jia P, Assadzadeh A, Flanagan J, Schumacher A, Wang S C, Petronis A. Epigenomic profiling reveals DNA-methylation changes associated with major psychosis. Am J Hum Genet. 2008 March; 82(3):696-711.
Shen L, Kondo Y, Guo Y, Zhang J, Zhang L, Ahmed S, Shu J, Chen X, Waterland R A, Issa J P. Genome-wide profiling of DNA methylation reveals a class of normally methylated CpG island promoters. PLoS Genet. 2007 October; 3(10):2023-36. Epub 2007 Sep. 10.
Matsuyama T. Epigenetics: use of in silico genome scanning by the virtual image restriction landmark method. FEBS J. 2008 April; 275(8):1607. Epub 2008 Mar. 7.
Wang S S, Smiraglia D J, Wu Y Z, Ghosh S, Rader J S, Cho K R, Bonfiglio T A, Nayar R, Plass C, Sherman M E. Identification of novel methylation markers in cervical cancer using restriction landmark genomic scanning. Cancer Res. 2008 Apr. 1; 68(7):2489-97.
Shivapurkar N, Stastny V, Xie Y, Prinsen C, Frenkel E, Czerniak B, Thunnissen F B, Minna J D, Gazdar A F. Differential Methylation of a Short CpG-Rich Sequence within Exon 1 of TCF21 Gene: A Promising Cancer Biomarker Assay. Cancer Epidemiol Biomarkers Prey. 2008 April; 17(4):995-1000.

1. A method for detecting cancer or an increased risk of having cancer in a subject, comprising the steps of:
(a) isolating a sample from said subject, wherein said sample comprises DNA;
(b) measuring a value of global DNA methylation index in said sample; and
(c) comparing said value of global DNA methylation index in said sample to a Standard value of global DNA methylation index; whereby if said value of global DNA methylation in said sample is lower than said Standard value of global DNA methylation index, then said subject has cancer or has an increased risk of having cancer.

2. The method of claim 1, wherein said standard value of global DNA methylation index is taken from a cancer-free subject or a pool of cancer-free subjects.

3. The method of claim 1, wherein said sample is a blood sample, saliva sample, tears sample, urine sample, cervical smear, ductal lavage fluid sample, cerebrospinal fluid sample, lymph fluid sample, serosal fluid, bile sample, stool sample, tumor sample, tissue sample, or any combination thereof.

4. The method of claim 1, wherein said cancer is a solid tumor or a hematological tumor.

5. The method of claim 1, wherein said cancer is a liver cancer, oral cancer, prostate cancer, lung cancer, colon cancer, bladder cancer or breast cancer.

6. The method of claim 1, further comprising the steps of:
(d) obtaining a sample from said subject, wherein said sample comprises histone H4;
(e) measuring a value of global histone H4 acetylation index and a value of global histone H4 trimethylation index in said sample; and
(f) comparing said value of global histone H4 acetylation index and said value of global histone H4 trimethylation index in said sample to a standard value of global histone H4 acetylation index and a standard value of global histone H4 trimethylation index, respectively, whereby if said value of global DNA methylation in said sample is lower than said standard value of global DNA methylation index; and said value of global histone H4 acetylation index and said value of global histone H4 trimethylation index in said sample are lower than said standard value of global histone H4 acetylation index and said standard value of global histone H4 trimethylation index, then said subject has cancer or an increased risk of having cancer.

7. The method of claim 6, wherein said sample comprising DNA is the same sample as said sample comprising histone H4.

8. The method of claim 6, wherein said sample comprising DNA is a different sample than said sample comprising histone H4.

9. The method of claim 6, wherein said standard value of global histone H4 acetylation index and/or said standard value of global histone H4 trimethylation index are taken from a cancer-free subject or a pool of cancer-free subjects.

10. The method of claim 6, wherein said histone H4 acetylation comprises histone H4 Lys16 acetylation.

11. The method of claim 6, wherein said histone H4 trimethylation comprises histone Lys20 trimethylation.

12-14. (canceled)

15. The method of claim 1, further comprising the steps of:
(a) isolating a sample from said subject, wherein said sample comprises histone H4;
(b) measuring a value of global histone H4 acetylation index and a value of global histone H4 trimethylation index in said sample; and
(c) comparing said value of global histone H4 acetylation index and said value of global histone H4 trimethylation index in said sample to a standard value of global histone H4 acetylation index and a standard value of global histone H4 trimethylation index, respectively, whereby if said value of global histone H4 acetylation index and said value of global histone H4 trimethylation index in said sample are lower than said standard value of global histone H4 acetylation index and said standard value of global histone H4 trimethylation index, then said subject has cancer or an increased risk of having cancer.

16. The method of claim 15, wherein said standard value of global histone H4 acetylation index and/or said standard value of global histone H4 trimethylation index are taken from a subject without increased risk of cancer, a pool of subjects without increased risk of cancer or a healthy subject or pool of healthy subjects.

17. The method of claim 15, wherein said histone H4 acetylation is histone H4 Lys16 acetylation.

18. The method of claim 15, wherein said histone H4 trimethylation is histone Lys20 trimethylation.

19. The method of claim 15, wherein said sample is a blood sample, saliva sample, tears sample, urine sample, cervical smear, ductal lavage fluid sample, cerebrospinal fluid sample, lymph fluid sample, serosal fluid, bile sample, stool sample, tumor sample, tissue sample, or any combination thereof.

20. The method of claim 15, wherein said cancer is a solid tumor or a hematological tumor.

21. The method of claim 15, wherein said cancer is a liver cancer, oral cancer, prostate cancer, lung cancer, colon cancer, bladder cancer or breast cancer.

22. A method of detecting an epigenetic change in a subject, comprising the steps of:
(a) isolating a sample from said subject, wherein said sample comprises DNA;
(b) measuring a value of global DNA methylation index in said sample; and
(c) comparing said value of global DNA methylation index in said sample to a standard value of global DNA methylation index, whereby if said value of global DNA methylation index in said sample is lower than or higher than said standard value of global DNA methylation index, then said subject has an epigenetic change.

23-24. (canceled)

25. A method of detecting an epigenetic change in a subject, comprising the steps of:
(a) isolating a sample from said subject, wherein said sample comprises DNA; (b) measuring a value of global DNA methylation index in said sample;
(c) comparing said value of global DNA methylation index in said sample to a standard value of global DNA methylation index;
(d) obtaining a sample from said subject, wherein said sample comprises histone H4;
(e) measuring a value of global histone H4 acetylation index and a value of global histone H4 trimethylation index in said sample; and
(f) comparing said value of global histone H4 acetylation index and said value of global histone H4 trimethylation index in said sample to a standard value of global histone H4 acetylation index and a standard value of global histone H4 trimethylation index, respectively, whereby if said value of global DNA methylation in said sample is lower than said standard value of global DNA methylation index; and said value of global histone H4 acetylation index and said value of global histone H4 trimethylation index in said sample are lower than said standard value of global histone H4 acetylation index and said standard value of global histone H4 trimethylation index, then said subject has cancer or an increased risk of having cancer.

26-31. (canceled)

32. The method of claim 22 or 25, wherein said epigenetic change in said subject indicates that said subject has or is at increased risk of having one or more of the following diseases: a cancer, an autoimmune disease, a neurodegenerative disease, a heart disease, a behavioral disorder, a musculoskeletal disease, a bone disease, a joint disease, a cartilage disease, a foot disease, a muscular disease, a neurological disease, an environmental disease, a vascular disease, a metabolic syndrome, a neuromuscular disease, an occupational disease, and a disease recognized as the Status Syndrome.

33. The method of claim 22 or 25, wherein said epigenetic change can be used to monitor one or more of the following: (a) modifiable lifestyle and contextual effects that impinge on cancer and other chronic and acute diseases, and (b) the effectiveness of strategies and therapies used to modify lifestyle and contextual effects to prevent disease, foster wellness and enable health promotion.
